(19) United States
(12) Patent Application Publication
(10) Pub. No.: US 2021/0222122 A1
(43) Pub. Date: Jul. 22, 2021

(54) SYNERGISTIC TRANSCRIPTION FACTORS TO INDUCE HIGH RESISTANCE TRANSENDOTHELIAL BARRIER

(71) Applicant: Hoffmann-La Roche Inc., Little Falls, NJ (US)

(72) Inventors: Chad A. COWAN, Boston, MA (US); Claas Aiko MEYER, Allschwil (CH); Filip ROUDNICKY, Zurich (CH)

(73) Assignee: Hoffmann-La Roche Inc., Little Falls, NJ (US)

(21) Appl. No.: 17/226,974

(22) Filed: Apr. 9, 2021

Related U.S. Application Data
(63) Continuation of application No. PCT/EP2019/078132, filed on Oct. 17, 2019.

(30) Foreign Application Priority Data
Oct. 19, 2018 (EP) 18201446.4

(51) Int. Cl. C12N 5/071; C12N 15/85

(52) U.S. Cl. CPC C12N 5/069; C12N 2501/60; C12N 15/85

(57) ABSTRACT

This application relates to transcription factors capable of increasing transendothelial barrier integrity. Moreover, this application relates to the use of vectors encoding such transcription factors and cells comprising such vectors.

### CROSS REFERENCE TO RELATED APPLICATIONS

This application is a continuation of International Application No. PCT/EP2019/078132, filed Oct. 17, 2019, which claims benefit to European Patent Application No. 18201446.4, filed Oct. 19, 2018, all of which are hereby expressly incorporated by reference in their entirety as though fully set forth herein.

### FIELD OF THE INVENTION

This application relates to transcription factors capable of increasing transendothelial barrier integrity. Moreover, this application relates to the use of vectors encoding such transcription factors and cells comprising such vectors.

### BACKGROUND

Endothelial cell (EC) development starts early in development and goes through separate steps of development with each step generating more specialized EC types, ultimately with the organ vascularization generating fully functional and specialized ECs (Potente M, Makinen T. Nature reviews Molecular cell biology. 2017; 18(8):477-94, Dejana E, Hirschi K K. 2017; 8:14361). ECs of the blood-brain barrier (BBB) are specialized as they form very high-resistance barrier by expression of tight junctions (Dejana E, Tournier-Lasserve E, Weinstein B M. Developmental cell. 2009; 16(2):209-21). Transcription factors are one of the main regulators of gene expression and development and their role in early vascular development has been extensively studied (De Val S, Black B L. Developmental cell. 2009; 16(2):180-95), but the transcriptional regulation of endothelial cell development during organ-specific differentiation is poorly understood (e.g. BBB, (Dejana E. 2010; 107(8):943-52, Mizee M R, Wooldrik D, Lakeman K A, van het Hof B, Drexhage J A, Geerts D, et al. The Journal of neuroscience: the official journal of the Society for Neuroscience. 2013; 33(4):1660-71, Engelhardt B, Liebner S. 2014; 355(3):687-99)).

Current models that exist for modeling EC barrier in vitro are highly sophisticated and difficult to accurately reproduce, making them difficult to adapt for drug discovery. Thus there remains a need for robust cell culture methods suitable to generate large quantities of ECs capable of establishing high resistance in vitro transendothelial barrier integrity (TBI) as a model to study e.g., BBB in healthy and diseased conditions.

The present inventors have previously established a simple and scalable 6-day protocol to differentiate human pluripotent stem cells into functional endothelial cells (Patsch C, Challet-Meylan L, Thoma E C, Urich E, Heckel T, O'Sullivan J F, et al. Nature cell biology. 2015; 17(8):994-1003).

Here, the inventors provide synergistic combinations of transcription factors that can generate endothelial cells (ECs) capable of establishing a high resistance barrier in vitro. The herein described combinations of transcription factors can be used in straightforward and robust protocols to produce ECs capable of establishing high barrier integrity that can be used, inter alia, for disease modeling or for drug discovery or toxicological studies.

### SUMMARY OF THE INVENTION

Provided is a method for producing cells capable of establishing high transendothelial electrical resistance (TEER), comprising the step of contacting the cells with at least one transcription factor, wherein a confluent monolayer of the cells establishes higher transendothelial electric resistance compared to a confluent monolayer of cells not contacted with the at least one transcription factor.

In one embodiment the at least one transcription factor is individually selected from the group consisting of TAL1, SOX18, FOXF2, SOX7, FOXC1, ETS1, KLF11, LMO2, and LEF1.

In one embodiment, the at least one transcription factor is selected from the group consisting of ETS1, SOX18 and SOX7, in particular wherein the transcription factors are ETS1, SOX18 and SOX7.

In one embodiment, the transcription factors are i) ETS1, SOX18, SOX7 and TAL1; or (ii) ETS1, SOX18, SOX7 and LEF1.

In one embodiment, isolated nucleic acids encoding the least one transcription factor are introduced into the cells.

In one embodiment, the isolated nucleic acids are comprised in at least one expression vector, in particular wherein the at least one expression vector is individually selected from the group consisting of a viral vector, a non-viral vector, and a plasmid vector.

In one embodiment, the cells are endothelial cells (ECs).

In one embodiment, provided is an expression vector comprising isolated nucleic acids encoding at least one transcription factor selected from the group consisting of TAL1, SOX18, FOXF2, SOX7, FOXC1, ETS1, KLF11, LMO2, and LEF1 In one embodiment, the vector is a viral vector, a non-viral vector, or a plasmid vector.

In one embodiment, the isolated nucleic acids encode at least one of the transcription factors selected from the group consisting of ETS1, SOX18 and SOX7, in particular the isolated nucleic acids encode the transcription factors ETS1, SOX18 and SOX7.

In one embodiment, the isolated nucleic acids encode the transcription factors (i) ETS1, SOX18, SOX7 and TAL1; or (ii) ETS1, SOX18, SOX7 and LEF1.

In one embodiment, provided is a cell comprising one or more of the expression vectors as herein described.

In one embodiment, the cell is a mammalian cell, in particular a human cell.

In one embodiment, provided is a method as herein described, wherein the cell capable of establishing high TEER is used for identifying a drug candidate capable of i) increasing in vivo transendothelial barrier integrity (TBI) or ii) decreasing in vivo TBI of endothelial cells (ECs). In one embodiment, the method comprises the steps of:
(a) providing a monolayer of cells capable of establishing high TEER;
(b) contacting the cells with the drug candidate;
(c) measuring in vitro TEER before and after contacting the cells with the drug candidate, or measuring in vitro TEER of the cells contacted with the drug candidate and in parallel measuring in vitro TEER of cells not contacted with the drug candidate;
   wherein (i) a higher in vitro TEER of the cells contacted with the drug candidate compared with the in vitro TEER of the cells not contacted with the drug candidate is indicative of a drug capable of increasing in vivo TBI of ECs, and (ii) a lower in vitro TEER of the cells contacted with the drug candidate compared with the in vitro TEER of the cells not contacted with the drug candidate is indicative of a drug capable of decreasing in vivo TBI of ECs.

### SHORT DESCRIPTION OF THE FIGURES

FIGS. 1A-1R. Identification of transcription factors that promote endothelial barrier resistance. Measurement of mean relative barrier resistance at 24 h after addition of 80 MOI adenovirus post-stabilization of the resistance measurement (at 10 h), averages are from 3 independent experiments (FIG. 1A). Real time measurement of EC barrier resistance for the transcription factors that had significant effect or a tendency to increase barrier resistance at 24 h was done with ECIS. The values were normalized to 10 h post treatment once the barrier measurements have stabilized. The lines denote the mean resistance (FIG. 1B). FITC-dextran measurement of permeability at 48 h post-transduction, averages are from 3 independent experiments (FIG. 1C). Relative mRNA expression of EC marker genes (FIGS. 1D, 1E, 1F), EC barrier junction forming genes (FIGS. 1G, 1H, 1I, 1J, 1K, 1L, 1M, 1N, 1O) and genes known to be important in EC barrier formation (FIGS. 1P, 1Q, 1R). Columns show means SD. *=p or FDR<0.05, **=p or FDR<0.01, **=p or FDR<0.001. All the treatments were performed in triplicates.

FIGS. 2A-2E. Overexpression of combination of transcription factors synergistically induce endothelial cell barrier resistance. Measurement of mean relative barrier resistance at 24 h after addition of 20 MOI adenovirus, post-stabilization of the resistance measurement (at 10 h), averages are from 3 independent experiments (FIG. 2A). FITC-dextran measurement of permeability at 48 h post-transduction, averages are from 3 independent experiments (FIG. 2B). Measurement of mean relative barrier resistance at 24 h after addition of combination of 4 adenovirus each with 20 MOI, post-stabilization of the resistance measurement (at 10 h), averages are from 3 independent experiments (FIG. 2C). Real-time measurements of EC barrier resistance for the combinations of TFs (each at 20 MOI). The lines denote the mean resistance and shading corresponds to standard deviation (FIG. 2D). FITC-dextran measurement of permeability at 48 h post-transduction; averages are from 3 independent experiments. Columns are mean SD (FIG. 2E).

### CITED REFERENCES

Potente M, Makinen T. Vascular heterogeneity and specialization in development and disease. Nature reviews Molecular cell biology. 2017; 18(8):477-94.
Dejana E, Hirschi K K. The molecular basis of endothelial cell plasticity. 2017; 8:14361.
Dejana E, Toumier-Lasserve E, Weinstein B M. The control of vascular integrity by endothelial cell junctions: molecular basis and pathological implications. Developmental cell. 2009; 16(2):209-21.
De Val S, Black B L. Transcriptional control of endothelial cell development. Developmental cell. 2009; 16(2):180-95.
Dejana E. The role of wnt signaling in physiological and pathological angiogenesis. Circulation research. 2010; 107(8):943-52.
Mizee M R, Wooldrik D, Lakeman K A, van het Hof B, Drexhage J A, Geerts D, et al. Retinoic acid induces blood-brain barrier development. The Journal of neuroscience: the official journal of the Society for Neuroscience. 2013; 33(4):1660-71.
Engelhardt B, Liebner S. Novel insights into the development and maintenance of the blood-brain barrier. Cell and tissue research. 2014; 355(3):687-99.
Englund M C, Caisander G, Noaksson K, Emanuelsson K, Lundin K, Bergh C, et al. The establishment of 20 different human embryonic stem cell lines and subclones; a report on derivation, culture, characterisation and banking. In vitro cellular & developmental biology Animal. 2010; 46(3-4):217-30.
Patsch C, Challet-Meylan L, Thoma E C, Urich E, Heckel T, O'Sullivan J F, et al. Generation of vascular endothelial and smooth muscle cells from human pluripotent stem cells. Nat Cell Biol. 2015; 17(8):994-1003.
Tam S J, Richmond D L, Kaminker J S, Modrusan Z, Martin-McNulty B, Cao T C, et al. Death receptors DR6 and TROY regulate brain vascular development. Developmental cell. 2012; 22(2):403-17.
Coppiello G, Collantes M, Sirerol-Piquer M S, Vandenwijngaert S, Schoors S, Swinnen M, et al. Meox2/Tcfl5 heterodimers program the heart capillary endothelium for cardiac fatty acid uptake. Circulation. 2015; 131(9):815-26.
Nolan D J, Ginsberg M, Israely E, Palikuqi B, Poulos M G, James D, et al. Molecular signatures of tissue-specific microvascular endothelial cell heterogeneity in organ maintenance and regeneration. Developmental cell. 2013; 26(2):204-19.
Ravasi T, Suzuki H, Cannistraci C V, Katayama S, Bajic V B, Tan K, et al. An atlas of combinatorial transcriptional regulation in mouse and man. Cell. 2010; 140(5):744-52. Laing E, Smith C P. RankProdIt: A web-interactive Rank Products analysis tool. BMC research notes. 2010; 3:221.
Vanlandewijck M, He L, Mae M A, Andrae J, Ando K, Del Gaudio F, et al. A molecular atlas of cell types and zonation in the brain vasculature. Nature. 2018; 554(7693):475-80.
Ben-Zvi A, Lacoste B, Kur E, Andreone B J, Mayshar Y, Yan H, et al. Mfsd2a is critical for the formation and function of the blood-brain barrier. Nature. 2014; 509:507.
Neph S, Stergachis A B, Reynolds A, Sandstrom R, Borenstein E, Stamatoyannopoulos J A. Circuitry and dynamics of human transcription factor regulatory networks. Cell. 2012; 150(6):1274-86.
Srinivasan B., Kolli A R, Esch M B, Abaci H E, Shuler M L, Hickman J J. J Lab Autom. 2015; 20(2):107-126.

### DETAILED DESCRIPTION

As used herein, the term “defined medium” or “chemically defined medium” refers to a cell culture medium in which all individual constituents and their respective concentrations are known. Defined media may contain recombinant and chemically defined constituents.

As used herein the term “differentiating”, “differentiation” and “differentiate” refers to one or more steps to convert a less-differentiated cell into a somatic cell, for example to convert a pluripotent stem cell into an EC. Differentiation of a pluripotent stem cell to a EC is achieved by methods described herein.

As used herein, “endothelial cells”, abbreviated “ECs”, are cells that express the specific surface marker CD144 (Cluster of Differentiation 144, also known as Cadherin 5, type 2 or vascular endothelial (VE)-cadherin, official symbol CDH5) and possess characteristics of endothelial cells, namely capillary-like tube formation, and the expression of one or more further surface markers selected from the group of, CD31 (Cluster of Differentiation 31, official symbol PECAM1), vWF (Von Willebrand factor, official symbol VWF), CD34 (Cluster of Differentiation 34, official symbol CD34), CD105 (Cluster of Differentiation 105, official symbol ENG), CD146 (Cluster of Differentiation 34, official symbol MCAM), and VEGFR-2 (kinase insert domain receptor (a type III receptor tyrosine kinase), official symbol KDR).

“Expansion medium” as used herein refers to any chemically defined medium useful for the expansion and passaging endothelial cells on a monolayer.

As used herein, the term “growth factor” means a biologically active polypeptide or a small molecule compound which causes cell proliferation, and includes both growth factors and their analogs.

“High-throughput screening” as used herein shall be understood to signify that a large number of different disease model conditions and/or chemical compounds can be analyzed and compared, parallel and/or sequential. Typically, such high-throughput screening is performed in multi-well microtiter plates, e.g., in a 96 well plate or a 384 well plate or plates with 1536 or 3456 wells.

“Induction medium” as used herein refers to any chemically defined medium useful for the induction of primed cells into CD144 positive (CD144+) endothelial cells on a monolayer.

A “monolayer of cells” as used herein means that the cells are provided as individual cells which are attached to the adhesive substrate in one single film, as opposed to culturing non-confluent single cells and/or cell clumps (e.g., embryoid bodies) in which a solid mass of cells in multiple layers form various three dimensional formations attached to the adhesive substrate.

The term “nucleic acids” relates to compositions and/or sequences of bases comprising purine- and pyrimidine bases which are comprised by polynucleotides, whereby said bases represent the primary structure of nucleic acids encoding a polypeptide and/or protein (e.g., a transcription factor). The term nucleic acids is herein used synonymously with the term polynucleotide(s) and nucleic acid molecules. Herein, the term nucleic acids includes DNA, cDNA, genomic DNA, RNA, synthetic forms of DNA and mixed polymers comprising two or more of these molecules. In addition, the term nucleic acids includes both, sense and antisense strands. Moreover, the herein described nucleic acids may contain non-natural or derivatized nucleotide bases, as will be readily appreciated by those skilled in the art.

By “isolated nucleic acids” molecule or “isolated polynucleotides” is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location.

“Nucleic acids encoding at least one transcription factor” refers to one or more nucleic acid molecules encoding a transcription factor of the invention (or fragments and/or mutants thereof), including such nucleic acid molecule(s) in a single vector or separate vectors, and such nucleic acid molecule(s) present at one or more locations in a host cell.

“Pluripotency medium” as used herein refers to any chemically defined medium useful for the attachment of pluripotent stem cells as single cells on a monolayer while maintaining their pluripotency. Useful pluripotency media are well known in the art also also described herein. In particular embodiments as described herein, the pluripotency medium contains at least one of the following growth factors: basic fibroblast growth factor (bFGF, also depicted as Fibroblast Growth Factor 2, FGF2) and transforming growth factor β (TGFβ).

As used herein, the term “reprogramming” refers to one or more steps needed to convert a somatic cell to a less-differentiated cell, for example for converting a fibroblast cell, adipocytes, keratinocytes or leucocyte into a pluripotent stem cell. “Reprogrammed” cells refer to cells derived by reprogramming somatic cells as described herein.

The term “small molecule”, or “small compound”, or “small molecule compound” as used herein, refers to organic or inorganic molecules either synthesized or found in nature, generally having a molecular weight less than 10,000 grams per mole, optionally less than 5,000 grams per mole, and optionally less than 2,000 grams per mole.

The term “somatic cell” as used herein refers to any cell forming the body of an organism that are not germline cells (e.g., sperm and ova, the cells from which they are made (gametocytes)) and undifferentiated stem cells.

The term “stem cell” as used herein refers to a cell that has the ability for self-renewal. An “undifferentiated stem cell” as used herein refers to a stem cell that has the ability to differentiate into a diverse range of cell types. As used herein, “pluripotent stem cells” refers to a stem cell that can give rise to cells of multiple cell types. Pluripotent stem cells (PSCs) include human embryonic stem cells (hESCs) and human induced pluripotent stem cells (hiPSCs). Human induced pluripotent stem cells can be derived from reprogrammed somatic cells, e.g. by transduction of four defined factors (Sox2, Oct4, Klf4, c-Myc) by methods known in the art and further described herein. Said human somatic cells can be obtained from a healthy individual or from a patient. These donor cells can be obtained from any suitable source. Preferred herein are sources that allow isolation of donor cells without invasive procedures on the human body, for example human skin cells, blood cells or cells obtainable from urine samples.

Although human cells are preferred, the methods as herein described are also applicable to non-human cells, such as primate, rodent (e.g. rat, mouse, rabbit) and dog cells.

As used herein, the term “high resistance transendothelial barrier”, refers to a functional hallmark of endothelial cells in vitro and in vivo. Endothelial cells (ECs) act as a semi-selective barrier between the vessel lumen and surrounding tissue, controlling the passage of materials and the transit of white blood cells into and out of the bloodstream. The integrity of this high resistance transendothelial barrier is herein referred to as “transendothelial barrier integrity” or “TBI”. Loss of barrier function can be observed in healthy and disease conditions, e.g., would healing, vascularization as well as chronic inflammation coincide with temporary or permanent loss of TBI. TBI can be modeled in vitro by monolayers of ECs (e.g., EC cultures) produced under appropriate conditions as described herein (e.g., EC culture from pluripotent cells) and known in the art (e.g., short-term primary cell culture). TBI, e.g., in vitro TBI, can be measured with methods known in the art (e.g., measuring TEER and FITC-dextran permeability) and as herein described. As used herein, the term “in vitro TBI” refers to TBI of an in vitro EC culture wherein the TBI is measured across the cell monolayer in culture, e.g., between the culture vessel surface below the monolayer and the cell culture medium above the monolayer of cells (in a classical 2D cell culture setup). Accordingly, as used herein, the term “in vivo TBI” refers to the TBI of ECs in vivo, wherein the TBI is established and/or determined (e.g., measured) between a vessel lumen and the surrounding tissue.

As used herein, the term “transendothelial electric resistance” or “TEER” refers to a quantitative measurement of the integrity of tight junction dynamic in a cell culture of EC monolayers. TEER is a widely accepted parameter in the context of in vitro barrier model systems (Srinivasan B., Kolli A R, Esch M B, Abaci H E, Shuler M L, Hickman J J. J Lab Autom. 2015; 20(2):107-126). In one embodiment, the electrical resistance or impedance of a cellular monolayer is a quantitative measurement of barrier integrity. Being a non-invasive technique, TEER is particularly suitable for repeated or real-time measurements on short-term, mid-term or long-term cell cultures. TEER measurements can be based on measuring ohmic resistance or impedance across a suitable spectrum of frequencies. In one embodiment, a cellular monolayer is cultured on a semipermeable filter insert which defines an upper (apical) and a lower (basolateral) compartment. In one embodiment, a first electrode placed in the apical compartment and a second electrode placed in the basolateral compartment are separated by the cellular monolayer. In one embodiment, an alternating current (AC) voltage signal with a square waveform is applied. In one embodiment, an AC at a frequency of 200-300 Hz is applied. In one embodiment, impedance over cells covering a circular gold electrode with a diameter of 250 μm is measured at 250 Hz.

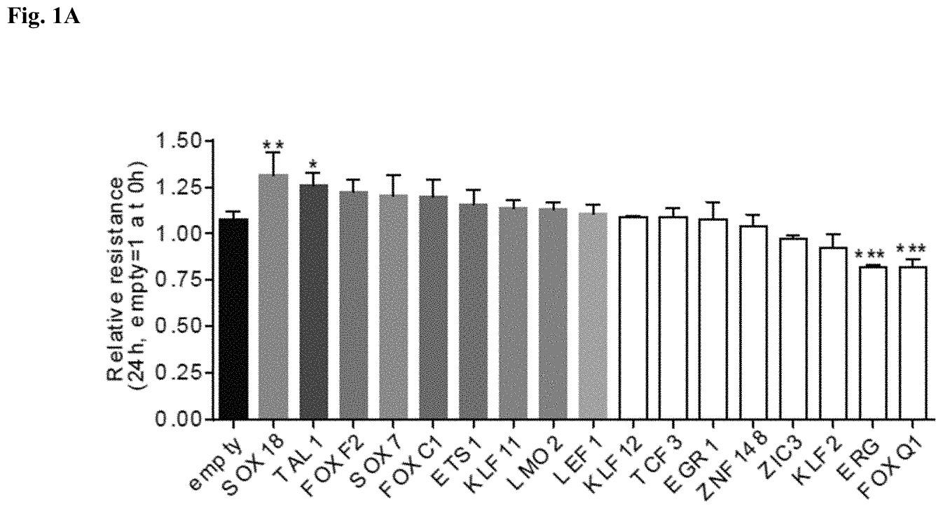

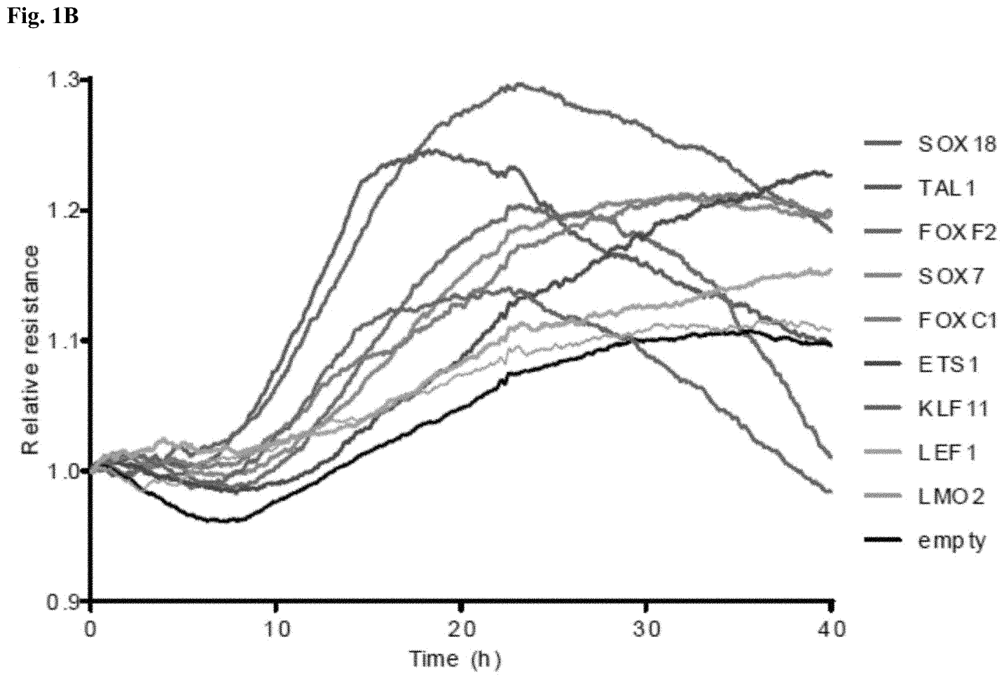

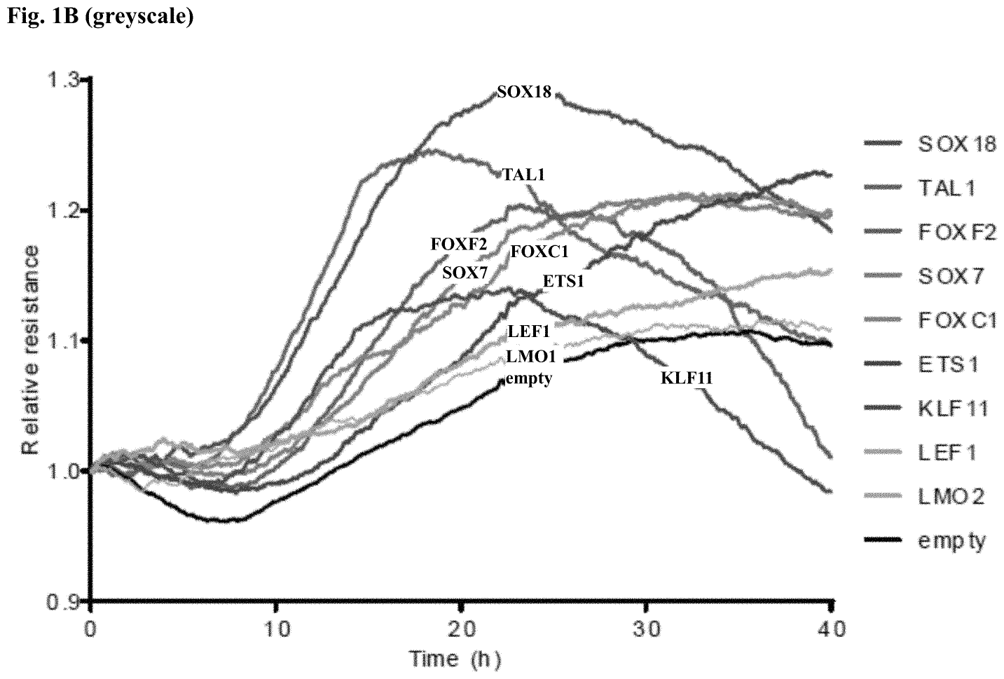

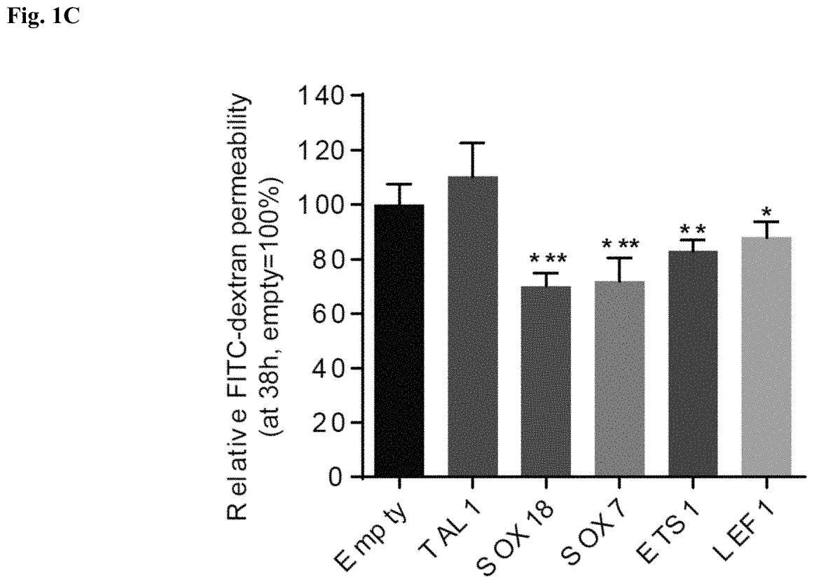

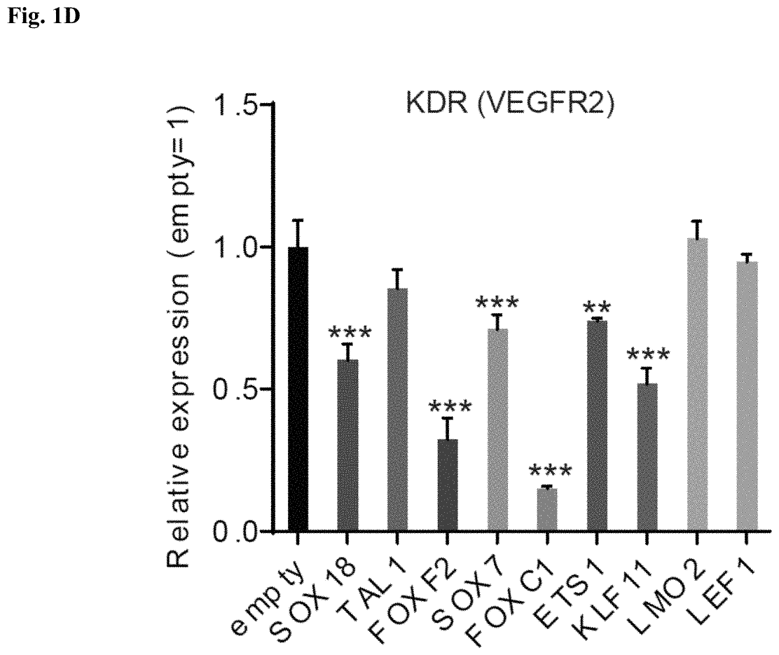

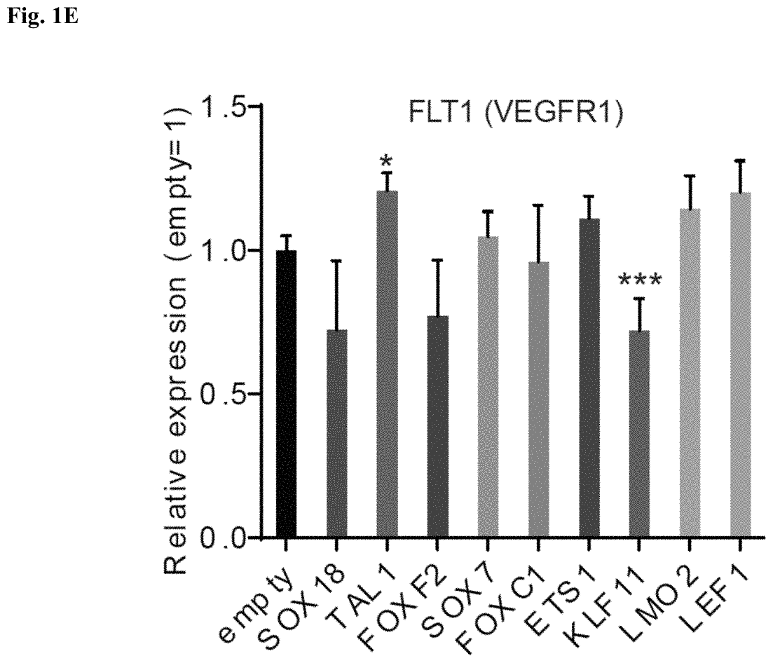

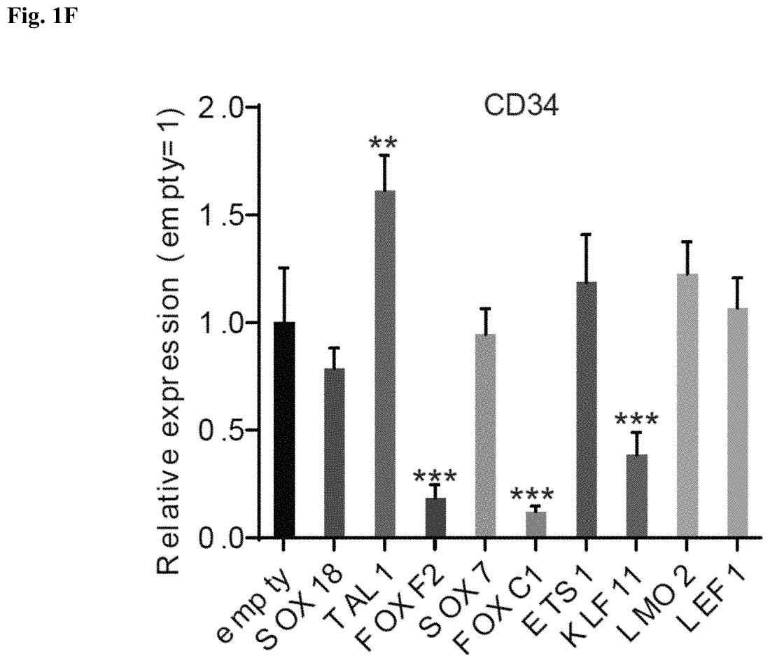

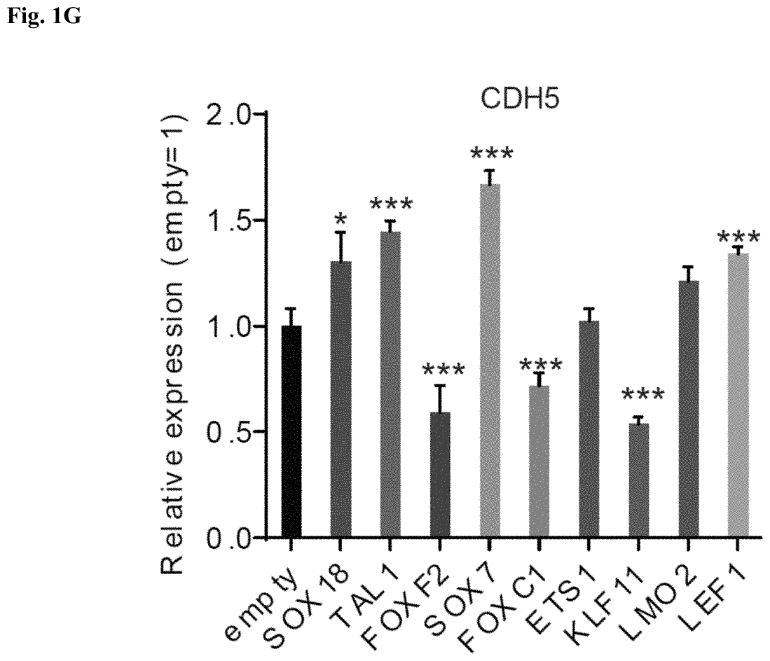

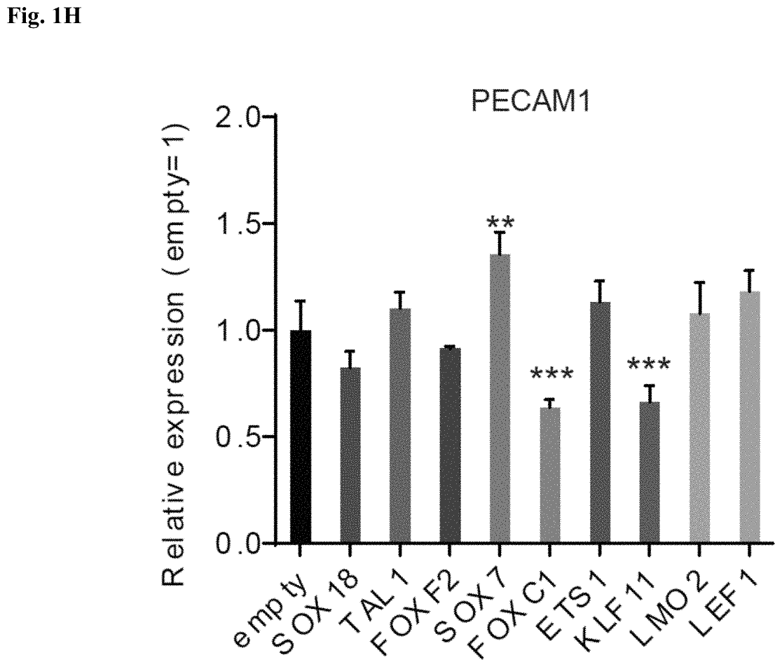

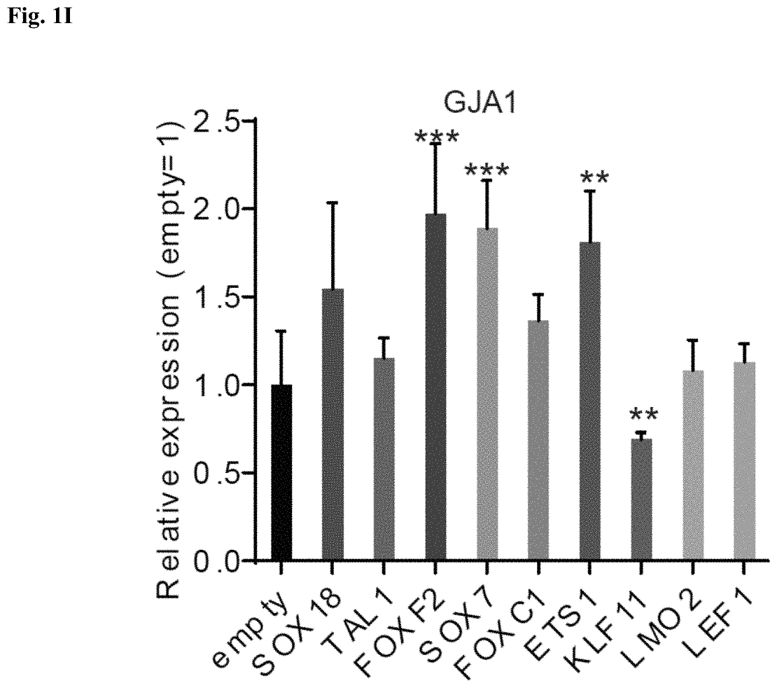

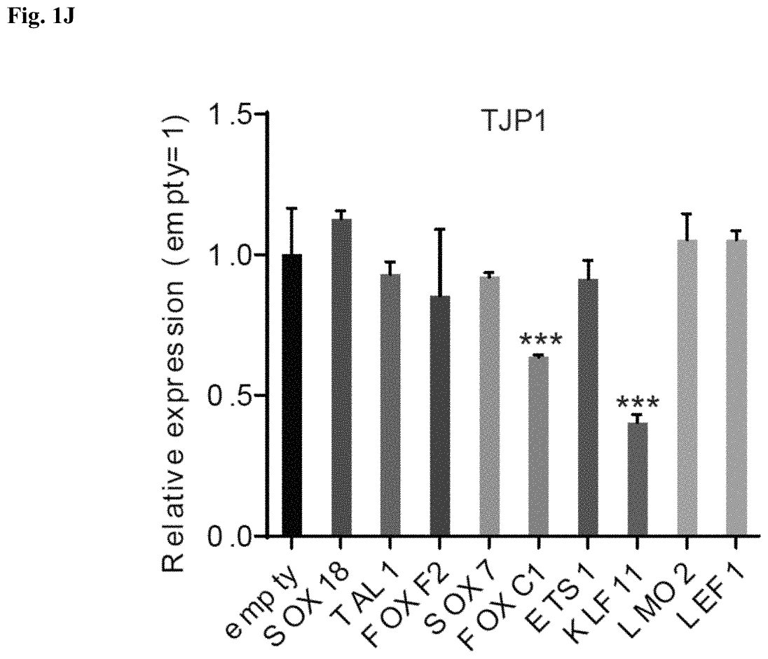

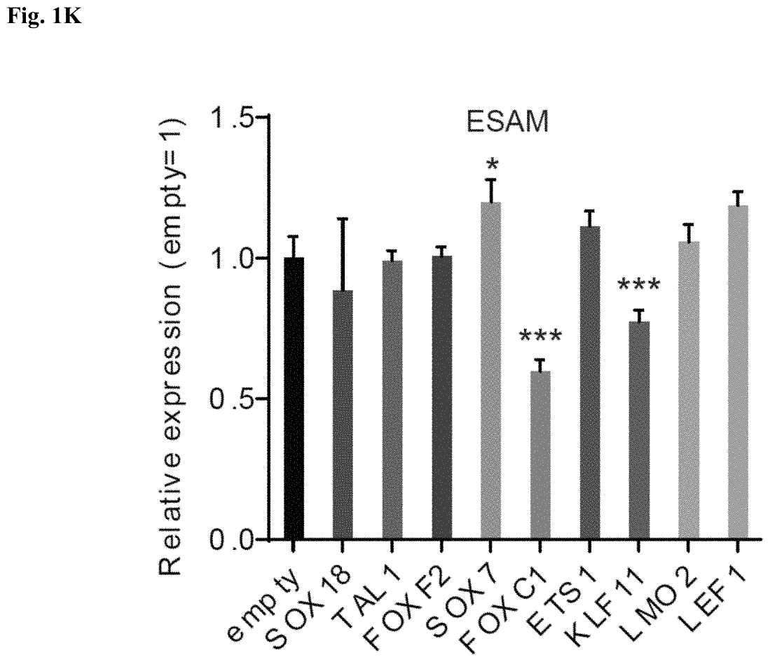

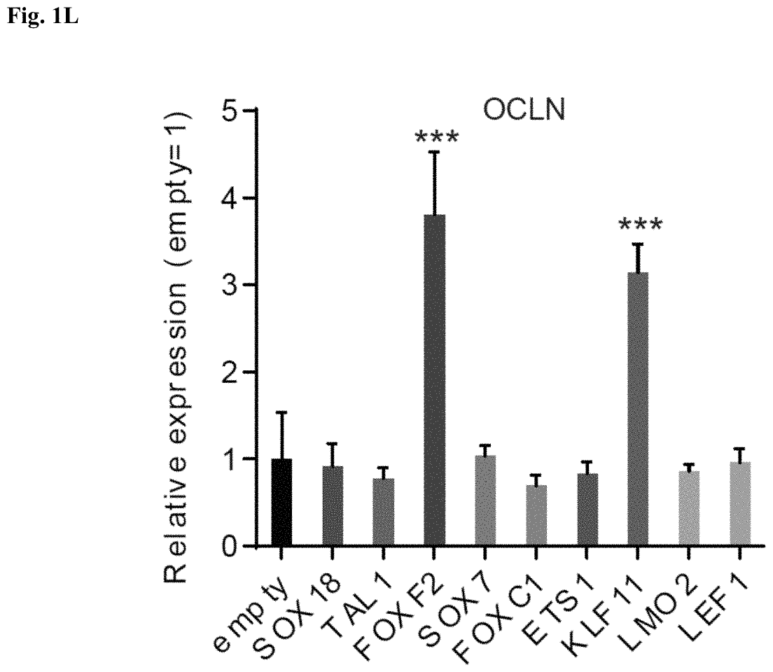

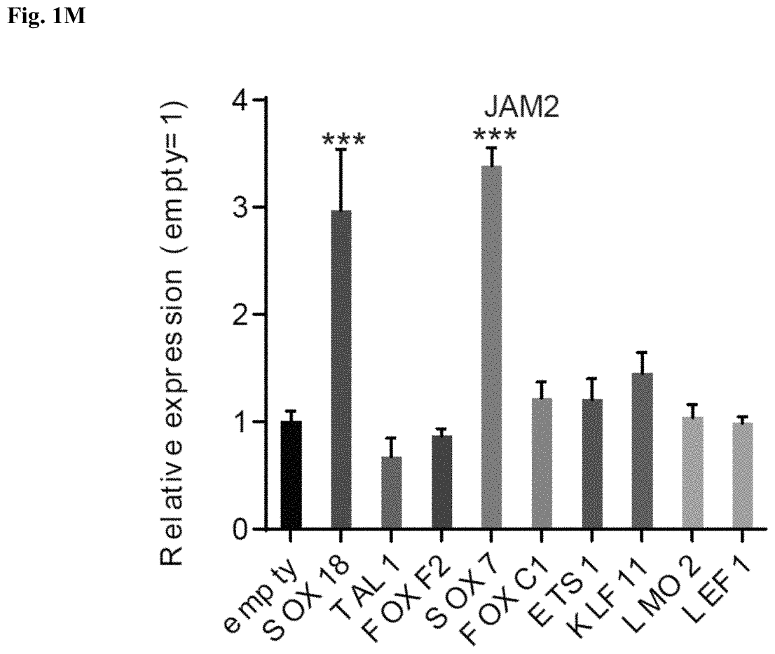

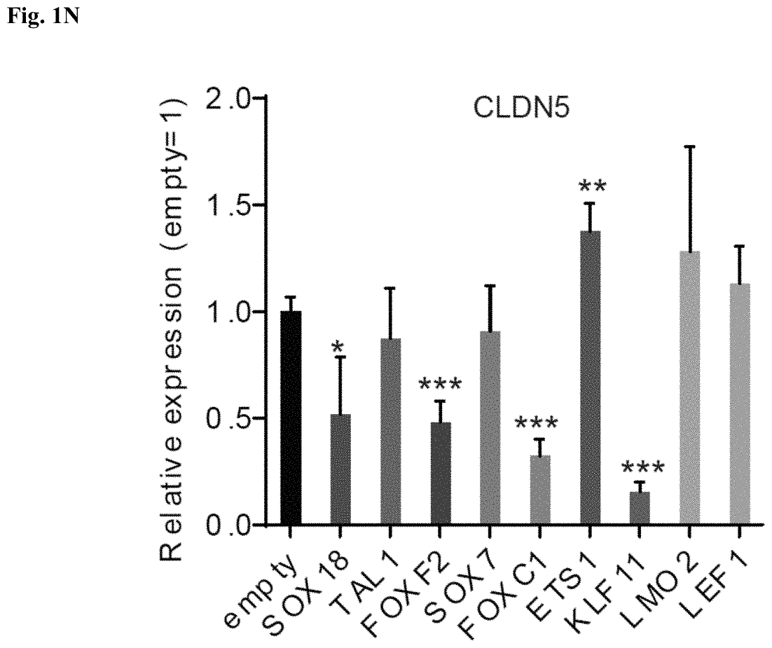

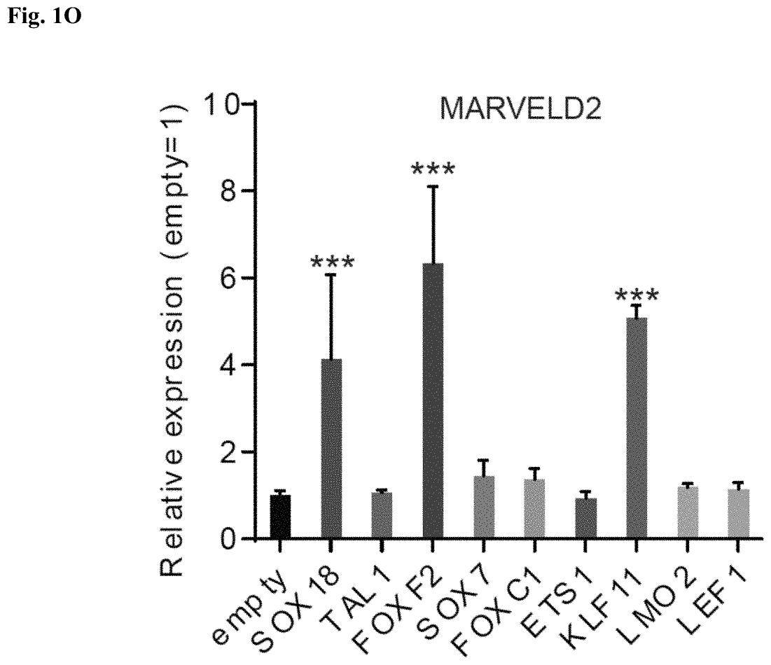

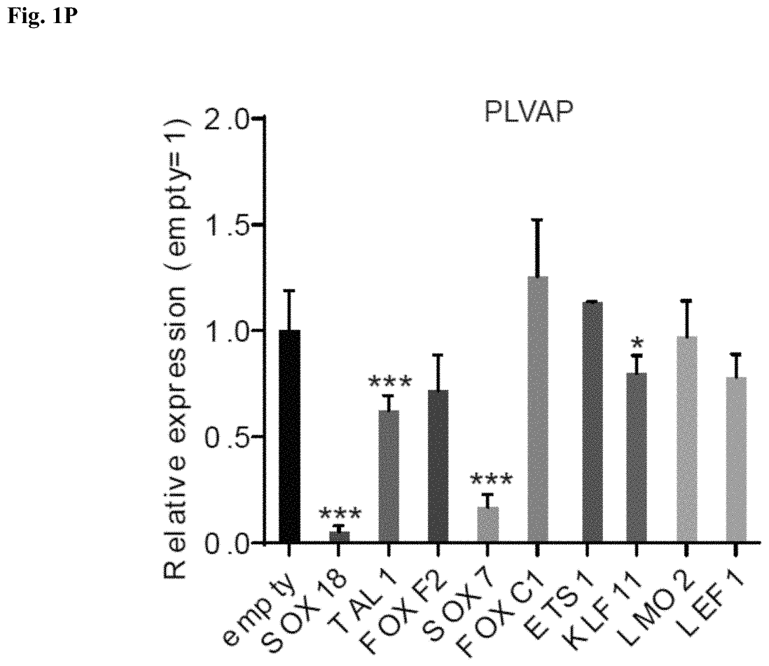

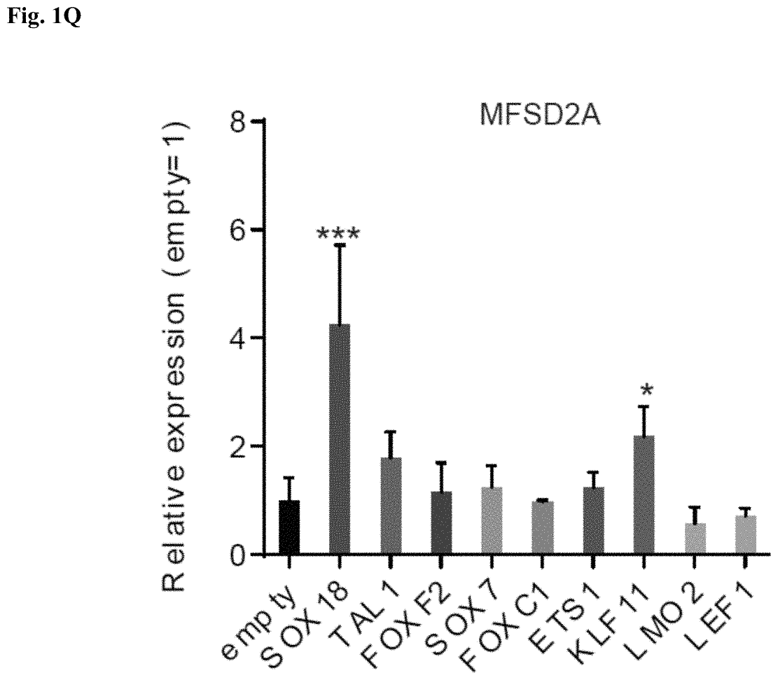

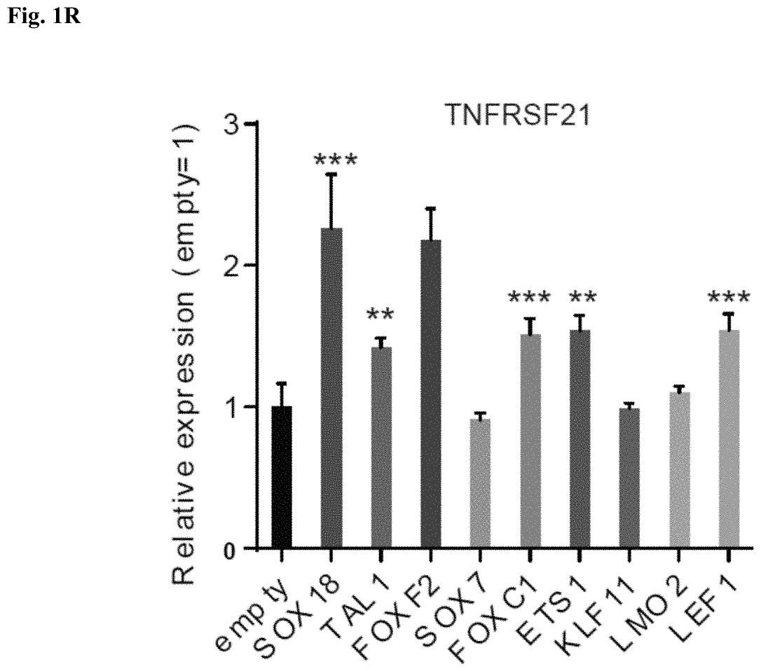

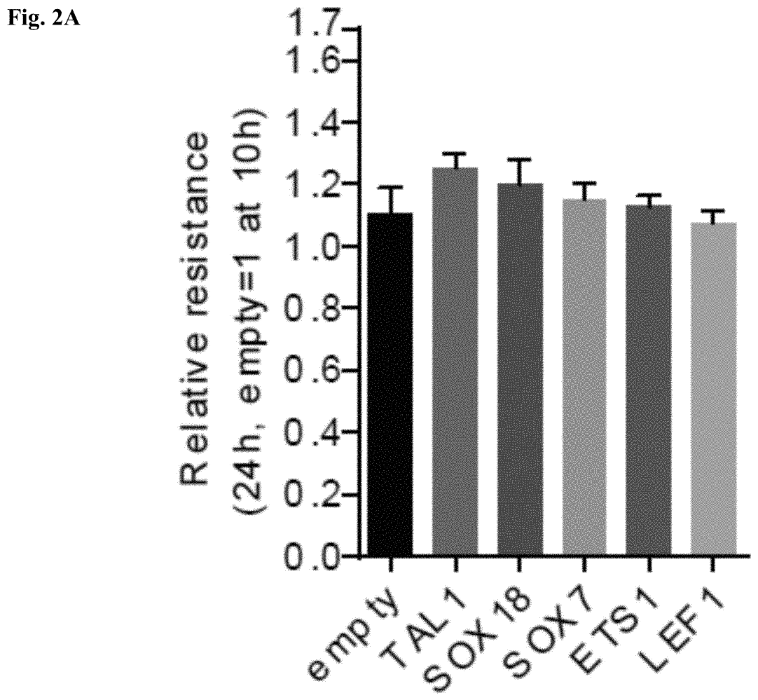

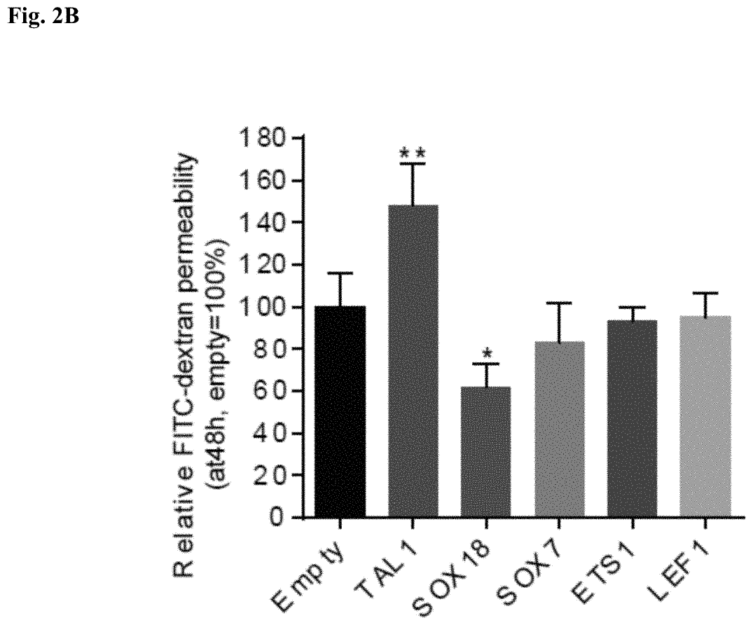

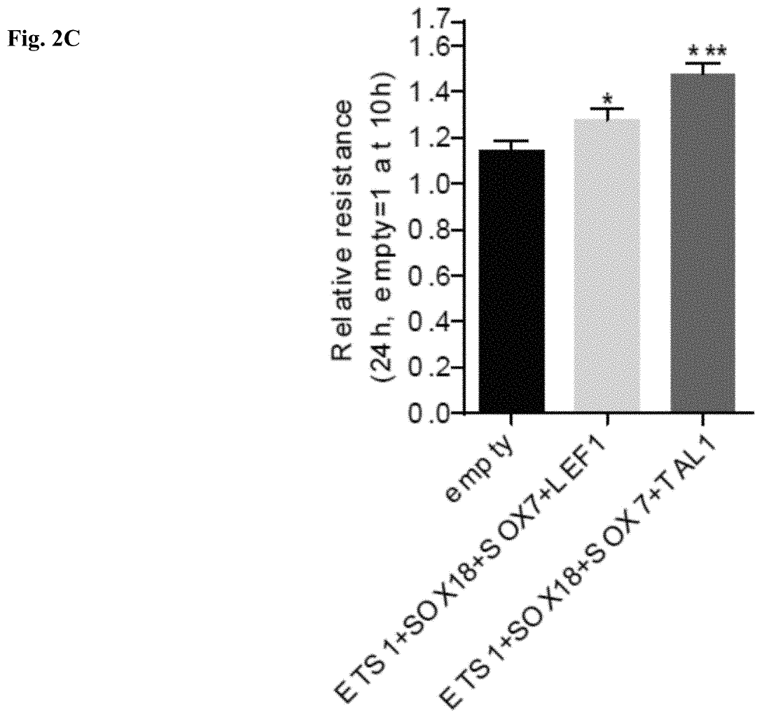

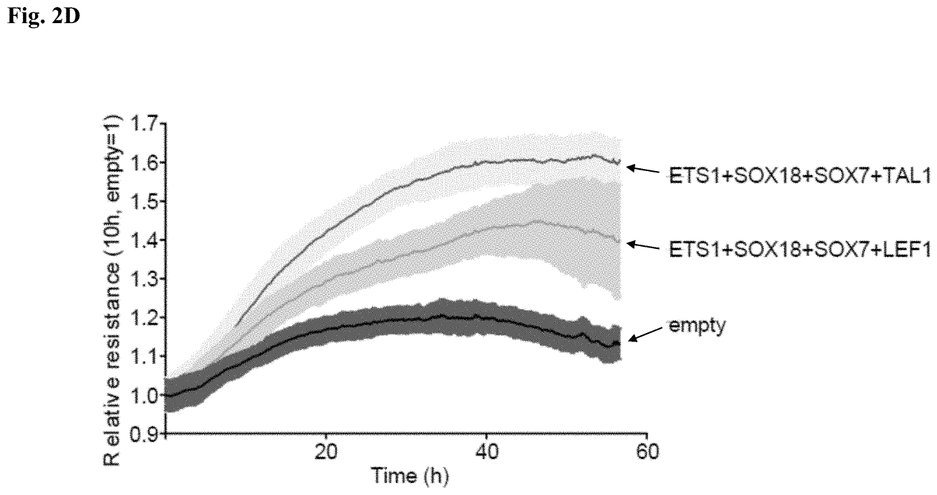

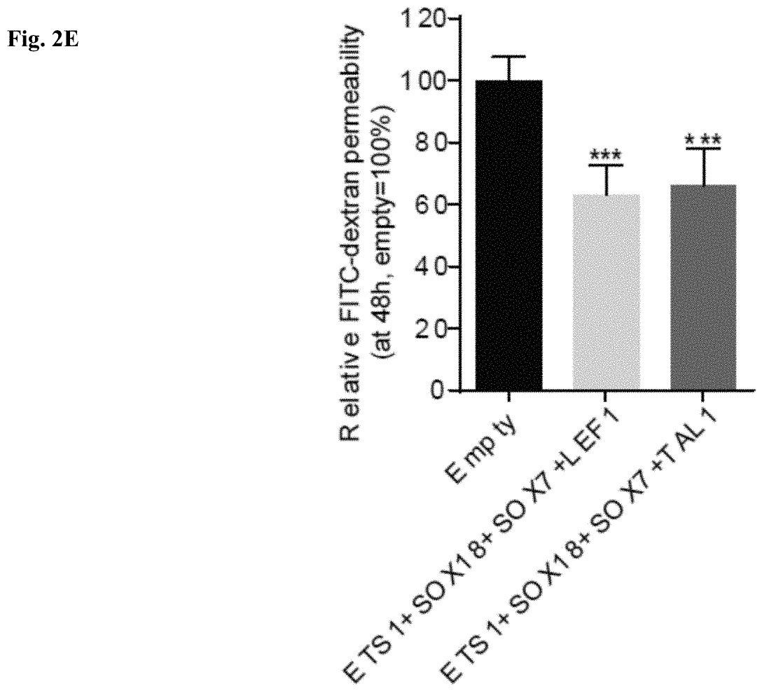

In this context, the present inventors surprisingly found that certain transcription factors, in particular combinations of transcription factors as herein described induce strong TBI in endothelial cells (ECs). Cells, in particular ECs, contacted with such transcription factors become capable of forming a high resistance transendothelial barrier restricting the passage of molecules and ions through, e.g., a monolayer of such cells. In this context, the present invention provides cells, in particular ECs, capable of forming/establishing a high resistance transendothelial barrier. Cells as herein described produced according to methods as herein described can, inter alia, be used to establish cell cultures, in particular a cell culture model of a healthy or diseased state wherein the transendothelial barrier integrity is a hallmark (e.g., TBI breakdown results from disease or BBB integrity). In one embodiment cell cultures as described herein are used in a drug development setting. An exemplary use of the cell cultures produced with the methods as described herein is prediction of in vivo response of cells/tissues to a drug candidate. As a proof of concept, ECs were contacted with combinations of transcription factors as herein described (e.g., TAL1, SOX18, FOXF2, SOX7, FOXC1, ETS1, KLF11, LMO2, LEF1, and combinations thereof), whereupon a striking increase of transendothelial electric resistance (TEER) was observed (see e.g., FIGS. 1B, 2C, 2D, 2E). TEER correlates with the movement of ions across e.g., a monolayer of cells and as such with TBI. In this context, high TEER correlates with an established high resistance transendothelial barrier as further described herein. This data and further data as herein provided demonstrates that specific transcription factors and combinations thereof as described herein are able to synergistically induce TBI (as e.g., measured by TEER or FITC-dextran permeability) and are useful to produce cells capable of forming/establishing a high resistance transendothelial barrier. The transcription factors as described herein, expression vectors encoding such transcription factors and cells comprising such expression vectors, inter alia, are useful to generate robust and meaningful models of ECs in health and disease. Such cell culture models can be used in methods to profile chemical libraries to find compounds that increase endothelial barrier integrity or prevent loss of barrier breakdown.

Cell are provided herein which are contacted with transcription factors. In response to the transcription factors, the cell establishes increased transendothelial barrier integrity which can be measured, e.g., by measuring TEER (in real-time) as known in the art and herein described.

Accordingly, cell contacted with transcription factors as herein described or combinations of transcription factors as herein described become capable of forming a high resistance transendothelial barrier. In a preferred embodiment, the cell(s) is are (an) endothelial cell(s). In a further embodiment, the cell(s) establishe(s) and/or maintains in vivo endothelial cell character in response to contacting with transcription factors as herein described.

In one embodiment, the cells are contacted with at least one transcription factor. In one embodiment, the transcription factor is selected from the group consisting of TAL1, SOX18, FOXF2, SOX7, FOXC1, ETS1, KLF11, LMO2, and LEF1. In one embodiment, the cells are contacted with two transcription factors selected from the group consisting of TAL1, SOX18, FOXF2, SOX7, FOXC1, ETS1, KLF11, LMO2, and LEF1. In one embodiment, the cells are contacted with three transcription factors selected from the group consisting of TAL1, SOX18, FOXF2, SOX7, FOXC1, ETS1, KLF11, LMO2, and LEF1. In one embodiment, the cells are contacted with four transcription factors selected from the group consisting of TAL1, SOX18, FOXF2, SOX7, FOXC1, ETS1, KLF11, LMO2, and LEF1. In one embodiment, the cells are contacted with five transcription factors are selected from the group consisting of TAL1, SOX18, FOXF2, SOX7, FOXC1, ETS1, KLF11, LMO2, and LEF1. In one particular embodiment, the transcription factors are ETS1, SOX18 and SOX7. In preferred embodiments, the transcription factors are (i) ETS1, SOX18, SOX7 and TAL; or (ii) ETS1, SOX18, SOX7 and LEF1, most preferred ETS1, SOX18, SOX7 and TAL.

In one embodiment, the at least one transcription factor is introduced into the cell. The transcription factors can be introduced into the cell, e.g. the EC, by methods known in the art and as herein described. In one embodiment, the transcription factors are encoded by isolated nucleic acids. In one embodiment, isolated nucleic acids encoding the at least one transcription factor are introduced into the cell. In one embodiment, the isolated nucleic acids comprise at least on polynucleotide. In one embodiment, the isolated nucleic acids encoding the at least one transcription factor are comprised in at least one expression vector. In one embodiment, the cell, e.g., the endothelial cell is contacted with at least one expression vector, in particular wherein the at least one expression vector is individually selected from the group consisting of a viral vector, a non-viral vector, and a plasmid vector. In one embodiment, 1, 2, 3, 4 or 5 expression vectors are introduced into the cell (e.g., the EC). In one embodiment, one expression vector, in particular a viral vector, is introduced into the cell. In one embodiment, two expression vectors are introduced into the cell, wherein the expression vectors are individually selected from the group consisting of a viral vector, a non-viral vector, and a plasmid vector. In one embodiment, three expression vectors are introduced into the cell, wherein the expression vectors are individually selected from the group consisting of a viral vector, a non-viral vector, and a plasmid vector. In one embodiment, four expression vectors are introduced into the cell, wherein the expression vectors are individually selected from the group consisting of a viral vector, a non-viral vector, and a plasmid vector. In one embodiment, five expression vectors are introduced into the cell, wherein the expression vectors are individually selected from the group consisting of a viral vector, a non-viral vector, and a plasmid vector. In a preferred embodiment the transcription factors are encoded by isolated nucleic acids which are comprised in one expression vector selected from the group consisting of a viral vector, a non-viral vector, and a plasmid vector, in particular a viral vector.

A further aspect of the present invention is isolated nucleic acids (polynucleotides) and vectors (e.g., expression vectors) encoding one or several transcription factor according to the present invention. In one embodiment, the expression vector comprises isolated nucleic acids encoding at least one transcription factor selected from the group consisting of TAL, SOX18, FOXF2, SOX7, FOXC1, ETS1, KLF11, LMO2, and LEF1. In one particular embodiment, the isolated nucleic acids encode the transcription factors ETS1, SOX18 and SOX7. In preferred embodiments, the isolated nucleic acids encode the transcription factors (i) ETS1, SOX18, SOX7 and TAL1; or (ii) ETS1, SOX18, SOX7 and LEF1, most preferred ETS1, SOX18, SOX7 and TAL1.

The polypeptide sequence(s) of such transcription factors are available in the UniProtKB/Swiss-Prot database and can be retrieved from http://www.uniprot.org/uniprot. Exemplary specific references for human transcription factors are herein provided. The sequence(s) of the (human) TAL1 (T-cell acute lymphocytic leukemia protein 1) can be obtained from the Swiss-Prot database entry P17542 (entry version 187, sequence version 2). The sequence(s) of the (human) SOX18 (Transcription factor SOX-18) can be obtained from the Swiss-Prot database entry P35713 (entry version 168, sequence version 2). The sequence(s) of the (human) FOXF2 (Forkhead box protein F2) can be obtained from the Swiss-Prot database entry Q12947 (entry version 149, sequence version 2). The sequence(s) of the (human) SOX7 (Transcription factor SOX-7) can be obtained from the Swiss-Prot database entry Q9BT81 (entry version 144, sequence version 1). The sequence(s) of the (human) FOXC1 (Forkhead box protein C1) can be obtained from the Swiss-Prot database entry Q12948 (entry version 184, sequence version 3). The sequence(s) of the (human) ETS1 (Protein C-ets-1) can be obtained from the Swiss-Prot database entry P14921 (entry version 203, sequence version 1). The sequence(s) of the (human) KLF11 (Krueppel-like factor 11) can be obtained from the Swiss-Prot database entry 014901 (entry version 171, sequence version 2). The sequence(s) of the (human) LMO2 (Rhombotin-2) can be obtained from the Swiss-Prot database entry P25791 (entry version 178, sequence version 1). The sequence(s) of the (human) LEF1 (Lymphoid enhancer-binding factor 1) can be obtained from the Swiss-Prot database entry Q9UJU2 (entry version 177, sequence version 1).

Isolated nucleic acids encoding at least one transcription factors as herein described, may be readily isolated and sequenced using conventional procedures (e.g., by using oligonucleotide probes that are capable of binding specifically to genes transcription factors as herein described) or produced by recombinant methods or obtained by chemical synthesis. Exemplary polynucleotide sequence(s) of transcription factors of the invention are available in the NCBI database and can be retrieved from www.ncbi.nlm.nih.gov. Exemplary specific references for human transcription factors are herein provided. A nucleic acid sequence encoding (human) TAL1 (T-cell acute lymphocytic leukemia protein 1) can be obtained from the NCBI database entry NP_001274276.1. A nucleic acid sequence encoding (human) SOX18 (Transcription factor SOX-18) can be obtained from the NCBI database entry NP_060889.1. A nucleic acid sequence encoding (human) FOXF2 (Forkhead box protein F2) can be obtained from the NCBI database entry NP_001443.1. A nucleic acid sequence encoding (human) SOX7 (Transcription factor SOX-7) can be obtained from the NCBI database entry NP_113627.1. A nucleic acid sequence encoding (human) FOXC1 (Forkhead box protein C1) can be obtained from the NCBI database entry NP_001444.2. A nucleic acid sequence encoding (human) ETS1 (Protein C-ets-1) can be obtained from the NCBI database entry NP_001137292.1. A nucleic acid sequence encoding (human) KLF11 (Krueppel-like factor 11) can be obtained from the NCBI database entry NP_001171187.1. A nucleic acid sequence encoding (human) LMO2 (Rhombotin-2) can be obtained from the NCBI database entry NP_001135787.1. A nucleic acid sequence encoding (human) LEF1 (Lymphoid enhancer-binding factor 1) can be obtained from the NCBI database entry NP_001124185.1.

Isolated nucleic acids encoding at least one transcription factors as herein described, can be isolated and inserted into one or more vectors for further cloning, introduction and/or expression in a host cell. Suitable vectors for transfecting or transducing, i.e. transforming, (eukaryotic) cells are known in the art and herein described. The isolated nucleic acids encoding one or several transcription factors of the invention may be under the control of regulatory sequences. For example, promoters, transcriptional enhancers and/or sequences which allow for induced expression of the transcription factors of the invention may be employed. In the context of the present invention, the isolated nucleic acids are expressed under the control of constitutive or inducible promoter. Suitable promoters are e.g., the CMV promoter, the UbiC promoter PGK, the EF1A promoter, the CAGG promoter, the SV40 promoter, the COPIA promoter, the ACT5C promoter, the TRE promoter, the Oct3/4 promoter, or the Nanog promoter. The present invention therefore also relates to (a) vector(s) comprising the isolated nucleic acids described in the present invention. In certain embodiments, the vector(s) are viral vectors, non-viral vectors, and plasmid vectors. In one embodiment, said vector is capable of transforming (e.g., transfecting or transducing) an eukaryotic cell. In one embodiment, the vector is capable of transducing an eukaryotic cell. Herein the term “vector” relates to a circular or linear nucleic acid molecule which can autonomously replicate in a host cell (i.e. in a transduced cell) into which it has been introduced. Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook et al. (loc cit.) and Ausubel, Current Protocols in Molecular Biology, Green Publishing Associates and Wiley Interscience, N.Y. (1989), (1994). Alternatively, the isolated nucleic acids (polynucleotides) and vectors of the invention can be reconstituted into liposomes for delivery to target cells. Furthermore, cloning vectors can be used to isolate individual sequences of DNA. Relevant sequences can be transferred into expression vectors where expression of a particular polypeptide is required. Typical cloning vectors include pBluescript SK, pGEM, pUC9, pBR322, pGA18 and pGBT9. Typical expression vectors include pTRE, pCAL-n-EK, pESP-1, pOP13CAT.

In this context, the invention also relates to (a) vector(s) comprising isolated nucleic acids (polynucleotides) which is (are) a regulatory sequence operably linked to said isolated nucleic acids encoding one or more transcription factors as defined herein. Such regulatory sequences (control elements) are known to the skilled person and may include a promoter, a splice cassette, translation initiation codon, translation and insertion site for introducing an insert into the vector(s). In the context of the present invention, said isolated nucleic acids are operatively linked to said expression control sequences allowing expression in eukaryotic or prokaryotic cells. It is envisaged that said vector(s) is (are) an expression vector(s) comprising the isolated nucleic acids encoding the transcription factor(s) as defined herein. Operably linked refers to a juxtaposition wherein the components so described are in a relationship permitting them to function in their intended manner. A control sequence operably linked to a coding sequence is ligated in such a way that expression of the coding sequence is achieved under conditions compatible with the control sequences. In case the control sequence is a promoter, it is obvious for a skilled person that double-stranded nucleic acid is preferably used. In one embodiment, the vector is polycistronic.

Expression comprises transcription of the isolated nucleic acids (polynucleotides) preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences encoding signal peptides capable of directing the polypeptide to a cellular compartment or secreting it into the medium may be added to the coding sequence of the recited nucleic acid sequence and are well known in the art.

In the context of the present invention, the expression control sequences will be eukaryotic promoter systems in vectors capable of transforming eukaryotic cells, but control sequences for prokaryotic cells may also be used. Once the vector has been incorporated into the appropriate cell, the cell is maintained under conditions suitable for expression of the nucleotide sequences. Additional regulatory elements may include transcriptional as well as translational enhancers. The above-described vectors of the invention can further comprise a selectable and/or scorable marker. Selectable marker genes useful for the selection of transformed cells, for example, npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (omithine decarboxylase) which confers resistance to the omithine decarboxylase inhibitor, 2-(difluoromethyl)-DL-omithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or ß-glucuronidase (Jefferson, EMBO J. 6 (1987), 3901-3907). This embodiment is particularly useful for simple and rapid screening of cells, tissues and organisms containing a recited vector.

The recited isolated nucleic acids and vector(s) may be designed for direct introduction or for introduction via liposomes, or viral vectors (e.g., adenoviral, retroviral) into the cell. In the context of the present invention, said cell is preferably an EC or a precursor of an EC.

In accordance with the above, the present invention relates to methods to derive vectors, particularly plasmids, cosmids and viral vectors used conventionally in genetic engineering that comprise isolated nucleic acids encoding at least one transcription factor as defined herein. In particular embodiment, the transcription factor(s) is (are) individually selected from the group consisting of TAL1, SOX18, FOXF2, SOX7, FOXC1, ETS1, KLF11, LMO2, and LEF1. In the context of the present invention, said vector is an expression vector and/or a gene transfer or targeting vector. Expression vectors derived from viruses such as retroviruses, vaccinia virus, adeno-associated virus, herpes virus, or bovine papilloma virus, may be used for delivery of the recited nucleic acid or vector into targeted cell populations.

The invention also provides for a cell transformed or transfected with a vector as described herein. Said cell may be produced by introducing at least one of the above described vectors into the cell or its precursor cell. The presence of said at least one vector as herein described indicates that the cell was transfected according to the present invention. The described isolated nucleic acids or vector(s) which is (are) introduced in the cell (e.g., the EC) or its precursor cell may either integrate into the genome of the cell or it may be maintained extrachromosomally.

In this context, provided are cells capable of establishing a high TEER, comprising the step of contacting the cells with at least one transcription factor, wherein a confluent monolayer of the cells establishes higher TEER compared to a confluent monolayer of cells not contacted with the at least one transcription factor. Without being bound to theory, a cell capable of forming a high resistance transendothelial barrier is a cell capable of establishing high transendothelial electrical resistance (TEER). In one embodiment, cells according to the present invention contacted with transcription factors as herein provided are capable of establishing high TEER upon forming a confluent cell monolayer. In one embodiment, a confluent monolayer of cells contacted with the at least one transcription factor is capable of establishing higher TEER compared to a confluent monolayer of cells not contacted with the at least one transcription factor. In one embodiment, a confluent monolayer of cells expressing the at least one transcription factor as herein described is capable of establishing higher TEER compared to a confluent monolayer of cells not expressing the at least one transcription factor. In one embodiment, a confluent monolayer of cells expressing at least one transcription factor selected from the group consisting of TAL1, SOX18, FOXF2, SOX7, FOXC1, ETS1, KLF11, LMO2, and LEF1 is capable of establishing higher TEER compared to a confluent monolayer of cells not expressing the at least one transcription factor. In one embodiment, the TEER of a confluent monolayer of cells expressing at least one transcription factor selected from the group consisting of TAL1, SOX18, FOXF2, SOX7, FOXC1, ETS1, KLF11, LMO2, and LEF1 is 1.1-fold, 1.2-fold, 1.3-fold, 1.4-fold, 1.5-fold, 1.6-fold, 1.7-fold, 1.8-fold, 1.9-fold, 2-fold, 5-fold or 10-fold higher compared to the TEER of a confluent monolayer of cells not expressing the at least one transcription factor. In one embodiment, the TEER of a confluent monolayer of cells expressing the transcriptions factor ETS1, SOX18 and SOX7 is 1.2-fold, 1.3-fold, 1.4-fold, 1.5-fold, 1.6-fold, 1.7-fold, 1.8-fold, 1.9-fold, 2-fold, 5-fold or 10-fold higher compared to the TEER of a confluent monolayer of cells not expressing the at least one transcription factor. In preferred embodiments, the transcription factors are (i) ETS1, SOX18, SOX7 and TAL1; or (ii) ETS1, SOX18, SOX7 and LEF1, most preferred ETS1, SOX18, SOX7 and TAL1.

In one embodiment, the cell capable of forming a high TEER is a mammalian cell. In one embodiment, the cell is a rodent cell, in particular a mouse cell or a rat cell. In a preferred embodiment, the cell is a human cell.

In a preferred embodiment, the cell is an endothelial cell (EC). ECs can be produced in vitro according to protocols known in the art. Particularly useful for the purpose of the present invention are ECs deriving from pluripotent stem cell. Pluripotent stem cells have self-renewal character and can be differentiated in all major cell types of the adult mammalian body. Pluripotent stem cells can be produced in large quantities under standardized cell culture conditions. Accordingly, in a preferred embodiment, the ECs are differentiated from pluripotent stem cells. In one embodiment, the ECs are differentiated from embryonic stem cells. In another embodiment, the ECs are differentiated from induced pluripotent stem cells (IPSCs). In one embodiment the IPSCs are generated from reprogrammed somatic cells. Reprogramming of somatic cells to IPSCs can be achieved by introducing specific genes involved in the maintenance of IPSC properties. Genes suitable for reprogramming of somatic cells to IPSCs include, but are not limited to Oct4, Sox2, Klf4 and C-Myc and combinations thereof. In one embodiment the genes for reprogramming are Oct4, Sox2, Klf4 and C-Myc.

Internal organs, skin, bones, blood and connective tissue are all made up of somatic cells. Somatic cells used to generate IPSCs include but are not limited to fibroblast cells, adipocytes and keratinocytes and can be obtained from skin biopsy. Other suitable somatic cells are leucocytes, erythroblasts cells obtained from blood samples or epithelial cells or other cells obtained from blood or urine samples and reprogrammed to IPSCs by the methods known in the art and as described herein. The somatic cells can be obtained from a healthy individual or from a diseased individual. In one embodiment, the somatic cells are derived from a subject (e.g., a human subject) suffering from a disease. In one embodiment, the disease is associated with vascular complications (e.g., similar to or identical to vascular complications associated with diabetic retinopathy and/or Wet AMD). The genes for reprogramming as described herein are introduced into somatic cells by methods known in the art, either by delivery into the cell via reprogramming vectors or by activation of said genes via small molecules. Methods for reprogramming comprise, inter alia, retroviruses, lentiviruses, adenoviruses, plasmids and transposons, microRNAs, small molecules, modified RNAs messenger RNAs and recombinant proteins. In one embodiment, a lentivirus is used for the delivery of genes as described herein. In another embodiment, Oct4, Sox2, Klf4 and C-Myc are delivered to the somatic cells using Sendai virus particles. In addition, the somatic cells can be cultured in the presence of at least one small molecule. In one embodiment, said small molecule comprises an inhibitor of the Rho-associated coiled-coil forming protein serine/threonine kinase (ROCK) family of protein kinases. Non-limiting examples of ROCK inhibitors comprise fasudil (1-(5-Isoquinolinesulfonyl) homopiperazine), Thiazovivin (N-Benzyl-2-(pyrimidin-4-ylamino) thiazole-4-carboxamide) and Y-27632 ((+)-(R)-trans-4-(1-aminoethyl)-N-(4-pyridyl) cyclo-hexanecarboxamide dihydrochloride).

Providing a defined monolayer of pluripotent stem cells is preferred for reproducibility and efficiency of the resulting cultures. In one embodiment, monolayers of pluripotent stem cells can be produced by enzymatically dissociating the cells into single cells and bringing them onto an adhesive substrate, such as pre-coated matrigel plates (e.g. BD Matrigel hESC-qualified from BD Bioscience, Geltrex hESC-qualified from Invitrogen, Synthemax from Corning). Examples of enzymes suitable for the dissociation into single cells include Accutase (Invitrogen), Trypsin (Invitrogen), TrypLe Express (Invitrogen). In one embodiment, 20000 to 60000 cells per cm2 are plated on the adhesive substrate. The medium used herein is a pluripotency medium which facilitates the attachment and growth of the pluripotent stem cells as single cells in a monolayer. In one embodiment, the pluripotency medium is a serum free medium supplemented with a small molecule inhibitor of the Rho-associated coiled-coil forming protein serine/threonine kinase (ROCK) family of protein kinases (herein referred to as ROCK kinase inhibitor).

Thus, in one embodiment, the method described herein comprises providing a monolayer of pluripotent stem cells in a pluripotency medium, wherein said pluripotency medium is a serum free medium supplemented with a ROCK kinase inhibitor.

Examples of serum-free media suitable for the attachment of the pluripotent stem cells to the substrate are mTeSR1 or TeSR2 from Stem Cell Technologies, Primate ES/iPS cell medium from ReproCELL and StemPro hESC SFM from Invitrogen, X—VIVO from Lonza. Examples of ROCK kinase inhibitor useful herein are Fasudil (1-(5-Isoquinolinesulfonyl)homopiperazine), Thiazovivin (N-Benzyl-2-(pyrimidin-4-ylamino)thiazole-4-carboxamide) and Y27632 ((+)-(R)-trans-4-(1-aminoethyl)-N-(4-pyridyl) cyclo-hexanecarboxamide dihydrochloride, e.g. Catalogue Number: 1254 from Tocris bioscience). In one embodiment, the pluripotency medium is a serum free medium supplemented with 2-20 μM Y27632, preferably 5-10 μM Y27632. In another embodiment the pluripotency medium is a serum free medium supplemented with 2-20 μM Fasudil. In another embodiment the pluripotency medium is a serum free medium supplemented with 0.2-10 μM Thiazovivin.

In one embodiment the method described herein comprises providing a monolayer of pluripotent stem cells in a pluripotency medium and growing said monolayer in the pluripotency medium for one day (24 hours). In another embodiment the method described herein comprises providing a monolayer of pluripotent stem cells in a pluripotency medium and growing said monolayer in the pluripotency medium for 18 hours to 30 hours, preferably for 23 to 25 hours.

In another embodiment the method described herein comprises providing a monolayer of pluripotent stem cells in a pluripotency medium, wherein said pluripotency medium is a serum-free medium supplemented with a ROCK kinase inhibitor, and growing said monolayer in the pluripotency medium for one day (24 hours). In another embodiment the method described herein comprises providing a monolayer of pluripotent stem cells in a pluripotency medium, wherein said pluripotency medium is a serum-free medium supplemented with a ROCK kinase inhibitor, and growing said monolayer in the pluripotency medium for 18 hours to 30 hours, preferably for 23 to 25 hours.

In one embodiment the cells are contacted with a priming medium to induce differentiation. In one embodiment, the cells are contacted with a priming medium supplemented with a small molecule that activates the Beta-catenin and/or Wnt signaling and/or Hedgehog (HH) signaling and inducing differentiation by incubating the primed cells in an induction medium. In one embodiment, the small molecule that activates the Beta-catenin and/or Wnt signaling and/or Hedgehog (HH) signaling is selected from the group of small molecule inhibitors of glycogen synthase kinase 3 (Gsk3a-b), small molecule inhibitors of CDC-like kinase 1 (Clk1-2-4, small molecule inhibitors of mitogen-activated protein kinase 15 (Mapk15), small molecule inhibitors of dual-specificity tyrosine-(Y)-phosphorylation regulated kinase (Dyrkla-b 4), small molecule inhibitors of cyclin-dependent kinase 16 (Pctk1-3 4), Smoothened (SMO) activators and modulators of the interaction between β-catenin (or γ-catenin) and the coactivator proteins CBP (CREB binding protein) and p300 (EA binding protein p300).

Preferably said glycogen synthase kinase 3 (Gsk3a-b) inhibitors are pyrrolidindione-based GSK3 inhibitors. “Pyrrolidindione-based GSK3 inhibitor” as used herein relates to selective cell permeable ATP-competitive inhibitors of GSK3a and GSK30 with low IC₅₀ values. In one embodiment the pyrrolidindione-based GSK3 inhibitor is selected from the group consisting of SB216763 (3-(2,4-Dichlorophenyl)-4-(1-methyl-1H-indol-3-yl)-1H-pyrrole-2,5-dione), SB415286 (3-[(3-Chloro-4-hydroxyphenyl)amino]-4-(2-nitrophenyl)-1H-pyrrol-2,5-dione), N⁶-{2-[4-(2,4-Dichloro-phenyl)-5-imidazol-1-yl-pyrimidin-2-ylamino]-ethyl}-3-nitro-pyridine-2,6-diamine 2HCl, 3-Imidazo[1,2-a]pyridin-3-yl-4-[2-(morpholine-4-carbonyl)-1,2,3,4-tetrahydro-[1,4]diazepino[6,7,1-hi]indol-7-yl]-pyrrole-2,5-dione, Kenpaullone (9-Bromo-7,12-dihydro-indolo[3,2-d][1]benzazepin-6(5H)-one), CHIR99021 (9-Bromo-7,12-dihydro-pyrido [3′,2′:2,3]azepino[4,5-b]indol-6(5H)-one) and (3-(3-Amino-phenyl)-4-(1-methyl-H-indol-3-yl)-pyrrole-2,5-dione (CP21R7, also referred to as “compound 21” herein; see e.g. L. Gong et al; Bioorganic& Medicinal Chemistry Letters 20 (2010), 1693-1696). In a preferred embodiment said pyrrolidindione-based GSK3 inhibitor is CP21R7.

In one embodiment said CDC-like kinase 1 (Clk1-2-4) inhibitor is selected from the group comprising benzothiazole and 3-Fluoro-N-[1-isopropyl-6-(1-methyl-piperidin-4-yloxy)-1,3-dihydro-benzoimidazol-(2E)-ylidene]-5-(4-methyl-H-pyrazole-3-sulfonyl)-benzamide.

In one embodiment said mitogen-activated protein kinase 15 (Mapk15) inhibitor is selected from the group comprising 4-(4-Fluorophenyl)-2-(4-methylsulfinylphenyl)-5-(4-pyridyl)-1H-imidazole (SB203580) and 5-Isoquinolinesulfonamide (H-89).

In one embodiment said dual-specificity tyrosine-(Y)-phosphorylation regulated kinase (Dyrkla-b 4) inhibitor is selected from the group comprising 6-[2-Amino-4-oxo-4H-thiazol-(5Z)-ylidenemethyl]-4-(tetrahydro-pyran-4-yloxy)-quinoline-3-carbonitrile.

In one embodiment said smoothened activator is Purmorphamine (2-(1-Naphthoxy)-6-(4-morpholinoanilino)-9-cyclohexylpurine.

Examples of modulators of the interaction between β-catenin (or γ-catenin) and the coactivator proteins CBP (CREB binding protein) and p300 (E1A binding protein p300) are IQ-1 (2-(4-Acetyl-phenylazo)-2-[3,3-dimethyl-3,4-dihydro-2H-isoquinolin-(1E)-ylidene]-acetamide, and ICG-001((6S,9aS)-6-(4-Hydroxy-benzyl)-8-naphthalen-1-ylmethyl-4,7-dioxo-hexahydro-pyrazino[1,2-a]pyrimidine-1-carboxylic acid benzylamide (WO 2007056593).

In one embodiment, the priming medium is supplemented with a small molecule inhibitor of Transforming growth factor beta (TGF β). In one embodiment, the small molecule inhibitor of TGF β is SB431542.

In one embodiment the method described herein comprises incubating said cells in a priming medium for about 2 to about 4 days (about 48 hours to about 96 hours). In one embodiment, step a) of the method described above comprises incubating said cells in a priming medium for about 3 days (about 72 hours).

In one embodiment said priming medium is a serum free medium supplemented with insulin, transferrin and progesterone. In one embodiment said serum free medium is supplemented with 10-50 μg/ml insulin, 10-100 μg/ml transferrin and 10-50 nM progesterone, preferably 30-50 μg/ml insulin, 20-50 μg/ml transferrin and 10-30 nM progesterone. Examples of serum-free media suitable for priming are N2B27 medium (N2B27 is a 1:1 mixture of DMEM/F12 (Gibco, Paisley, UK) supplemented with N2 and B27 (both from Gibco)), N3 medium (composed of DMEM/F12 (Gibco, Paisley, UK), 25 μg/ml insulin, 50 μg/ml transferrin, 30 nM sodium selenite, 20 nM progesterone, 100 nM putrescine (Sigma)), or NeuroCult® NS-A Proliferation medium (Stemcell Technologies). In one embodiment said priming medium is a serum free medium supplemented with insulin, transferrin, progesterone and a small molecule that activates the Beta-Catenin (cadherin-associated protein, beta 1; human gene name CTNNB1) pathway and/or the Wnt receptor signaling pathway and/or hedgehog (HH) signaling pathway. Preferably said small molecule is selected from the group comprising 3-(2,4-Dichlorophenyl)-4-(1-methyl-H-indol-3-yl)-1H-pyrrole-2,5-dione (SB216763), 3-[(3-Chloro-4-hydroxyphenyl)amino]-4-(2-nitrophenyl)-1H-pyrrol-2,5-dione (SB415286), N⁶-{2-[4-(2,4-Dichloro-phenyl)-5-imidazol-1-yl-pyrimidin-2-ylamino]-ethyl}-3-nitro-pyridine-2,6-diamine 2HCl, 3-Imidazo[1,2-a]pyridin-3-yl-4-[2-(morpholine-4-carbonyl)-1,2,3,4-tetrahydro-[1,4]diazepino[6,7,1-hi]indol-7-yl]-pyrrole-2,5-dione, 9-Bromo-7,12-dihydro-indolo[3,2-d][1]benzazepin-6(5H)-one (Kenpaullone), 9-Bromo-7,12-dihydro-pyrido[3′,2′:2,3]azepino[4,5-b]indol-6(5H)-one (CHIR99021), 3-(3-Amino-phenyl)-4-(1-methyl-H-indol-3-yl)-pyrrole-2,5-dione (CP21R7, also referred to as “compound 21” herein), benzothiazole, 3-Fluoro-N-[1-isopropyl-6-(1-methyl-piperidin-4-yloxy)-1,3-dihydro-benzoimidazol-(2E)-ylidene]-5-(4-methyl-TH-pyrazole-3-sulfonyl)-benzamide, 4-(4-Fluorophenyl)-2-(4-methylsulfinylphenyl)-5-(4-pyridyl)-1H-imidazole (SB203580), 5-Isoquinolinesulfonamide (H-89), 6-[2-Amino-4-oxo-4H-thiazol-(5Z)-ylidenemethyl]-4-(tetrahydro-pyran-4-yloxy)-quinoline-3-carbonitrile, 2-(1-Naphthoxy)-6-(4-morpholinoanilino)-9-cyclohexylpurine (Purmorphamine), 2-(4-Acetyl-phenylazo)-2-[3,3-dimethyl-3,4-dihydro-2H-isoquinolin-(1E)-ylidene]-acetamide (IQ-1), and ICG-001 ((6S,9aS)-6-(4-Hydroxy-benzyl)-8-naphthalen-1-ylmethyl-4,7-dioxo-hexahydro-pyrazino[1,2-a]pyrimidine-1-carboxylic acid benzylamide.

In another embodiment the method described herein comprises incubating said cells in a priming medium, wherein said priming medium is a serum-free medium supplemented with CP21R7 (3-(3-Amino-phenyl)-4-(1-methyl-1H-indol-3-yl)-pyrrole-2,5-dione). Preferably said priming medium is supplemented with 0.5-4 μM CP21R7 (3-(3-Amino-phenyl)-4-(1-methyl-1H-indol-3-yl)-pyrrole-2,5-dione), most preferably 1-2 μM CP21R7 (3-(3-Amino-phenyl)-4-(1-methyl-H-indol-3-yl)-pyrrole-2,5-dione). In another embodiment step a) of the method described above comprises incubating said cells in a priming medium, wherein said priming medium is a serum-free medium supplemented with CP21R7 (3-(3-Amino-phenyl)-4-(1-methyl-H-indol-3-yl)-pyrrole-2,5-dione), and growing said cells for 2 to 4 days (48 hours to 96 hours). In another embodiment step a) of the method described above comprises incubating said cells in a priming medium, wherein said priming medium is a serum-free medium supplemented with CP21R7 (3-(3-Amino-phenyl)-4-(1-methyl-H-indol-3-yl)-pyrrole-2,5-dione), and incubating said cells for three days (72 hours).

In one embodiment the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-H-indol-3-yl)-pyrrole-2,5-dione).

In one embodiment the priming medium additionally comprises recombinant bone morphogenic protein-4 (BMP4). In one preferred embodiment the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-H-indol-3-yl)-pyrrole-2,5-dione) and 10-50 ng/ml recombinant bone morphogenic protein-4 (BMP4). In one embodiment, the priming medium comprises 1 μM CP21R7 and 25 ng/ml BMP4.

In one embodiment the cells are contacted with an induction medium to proceed differentiation. For predominant induction of endothelial cells, said induction medium is supplemented with VEGF (=Vascular endothelial growth factor) or placenta-like growth factor 1 (PLGF-1) and a small molecule adenylate cyclase activator. In one embodiment said small molecule adenylate cyclase activator leads to the activation of PKA/PKI signaling pathway. In one embodiment, said small molecule adenylate activators are chosen from the group comprising Forskolin ((3R)-(6aalphaH)Dodecahydro-6beta,10alpha,10balpha-trihydroxy-3beta,4abeta,7,7, 10abeta-pentamethyl-1-oxo-3-vinyl-1H-naphtho[2,1-b]pyran-5beta-yl acetate), 8-Bromo-cAMP (8-Bromoadenosine-3′,5′-cyclic monophosphate) and Adrenomedullin. In one embodiment said induction medium is a serum free medium supplemented with human serum albumin, ethanolamine, transferrin, insulin and hydrocortisone. Examples of serum-free media suitable for the induction are StemPro-34 (Invitrogen, principal components: human serum albumin, lipid agents such as Human Ex-Cyte® and ethanolamine or a mixture thereof, human zinc insulin, hydrocortisone, iron-saturated transferring 2-mercaptoethanol, and D,L-tocopherol acetate, or derivatives or mixtures thereof) and X-VIVO 10 and 15 (Lonza).

In one embodiment, said induction medium is a serum-free medium supplemented with human serum albumin, ethanolamine, transferrin, insulin and hydrocortisone, and 1-10 μM Forskolin and 5-100 ng/ml VEGF-A. In another embodiment, the induction medium comprises StemPro-34 (from Invitrogen) supplemented with VEGF-A 30-70 ng/ml or placenta-like growth factor 1 (PLGF-1) 30-70 ng/ml.

In one embodiment the method described herein comprises inducing the differentiation into ECs by incubating said primed cells in an induction medium supplemented with VEGF-A or placenta-like growth factor 1 (PLGF-1) and a small molecule adenylate cyclase activator, wherein said small molecule adenylate cyclase activator is selected from the group of Forskolin, 8-Bromo-cAMP and Adrenomedullin. In one embodiment, the induction medium is a serum-free medium supplemented with 1-10 μM Forskolin and 5-100 ng/ml VEGF-A, preferably 2 μM Forskolin and 50 ng/ml VEGF-A

In another embodiment the method described herein comprises inducing the differentiation into ECs by incubating said primed cells in an induction medium supplemented with VEGF-A or placenta-like growth factor 1 (PLGF-1) and a small molecule adenylate cyclase activator for one day.

In another embodiment the method described herein comprises inducing the differentiation into ECs by incubating said primed cells in an induction medium supplemented with VEGF-A or placenta-like growth factor 1 (PLGF-1) and a small molecule adenylate cyclase activator for 18 hours to 48 hours, preferably for 22 hours to 36 hours.

In one embodiment the method described herein comprises incubating said cells the induction medium for about 18 hours to about 48 hours. In one embodiment the method described herein comprises incubating said cells in an induction medium for about 24 hours.

After priming and induction, the ECs can be further expanded to produce large quantities of cells. Accordingly, in a further embodiment, the method of the invention additionally comprises incubating the product of priming and induction under conditions suitable for proliferation of the ECs. Said conditions suitable for proliferation of the endothelial cells can further comprise harvesting of the cells positive for endothelial markers and expanding them in a chemically defined expansion medium. “Harvesting” as used herein relates to the enzymatical dissociation of the cells from the adhesive substrate and subsequent resuspension in new medium. In one preferred embodiment, cells are sorted after harvesting as herein described. In one embodiment said expansion medium is a serum free medium supplemented with VEGF-A. Examples of serum-free media suitable for the expansion of ECs are StemPro-34 (Invitrogen), EGM2 (Lonza) and DMEM/F12 (Invitrogen) supplemented with 8 ng/ml FGF-2, 50 ng/ml VEGF and 10 μM SB431542 (4-(4-Benzo[1,3]dioxol-5-yl-5-pyridin-2-yl-TH-imidazol-2-yl)-benzamide). Preferably, the ECs are cultured in adherent culturing conditions. In one embodiment, the expansion medium is supplemented with 5-100 ng/ml VEGF-A. In another embodiment, the expansion medium is StemPro-34 supplemented with 5-100 ng/ml VEGF-A, preferably 50 ng/ml.

The cells (e.g., ECs) obtained by the method described herein can be expanded for several passages and culturing is well characterized. It is possible to freeze and thaw aliquots of the ECs obtained by the method described herein reproducibly. Thawed cells can be further expanded as described herein to reach a desired number of cells which is particularly suitable to establish the throughput needed for compound screening.

In one embodiment of the present invention a method for generating patient specific or healthy individual specific ECs. This is particularly desirable for disease condition associated with a genetic mutation, however, a patient specific disease model can also be relevant where no genetic mutation is associated with the disease condition or in situations where a link to a genetic mutation is not known or should be established. Towards this end, human induced pluripotent stem cells (iPSCs) obtained from a patient or healthy individual are used in the method described herein. Said patient-specific human iPSCs can be obtained by methods known in the art and as further described herein by reprogramming somatic cells obtained from the patients or healthy individuals to pluripotent stem cells. For example, fibroblast cells, keratinocytes or adipocytes may be obtained by skin biopsy from the individual in need of treatment or from a healthy individual and reprogrammed to induced pluripotent stem cells by the methods known in the art and as further described herein. Other somatic cells suitable as a source for induced pluripotent stem cells are leucocytes cells obtained from blood samples or epithelial cells or other cells obtained from urine samples. The patient specific induced pluripotent stem cells are then differentiated to patient specific diseased or healthy ECs by the method described herein. In another aspect of the invention, a population of ECs produced by any of the foregoing methods is provided. Preferably, the population of ECs is patient specific, i.e. derived from iPSCs obtained from diseased individuals. In another embodiment said population of ECs is obtained from a healthy individual. Patient derived ECs represent a disease relevant in vitro model to study the pathophysiology of vascular complications for diseases like Diabetes Type-2 and Type-1, Wet AMD, Metabolic Syndrome and Severe Obesity. In one embodiment the ECs obtained by this method are used for screening for compounds that reverse, inhibit or prevent vascular complications caused by dysfunction of ECs, e.g. of vascular complications caused by diabetes Type-2 and Type-1, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity. Preferably, said ECs obtained by the method of the invention described herein are derived from diseased subjects. Differentiating ECs from diseased subjects represents a unique opportunity to early evaluate drug safety in a human background paradigm.

In another embodiment the ECs obtained by this method are used as an in vitro model of the blood-retinal barrier (BRB) and/or the blood brain barrier (BBB). The cells produced according to the methods of the present invention are useful to establish in vitro models of pathological or non-pathological conditions wherein the establishment or loss of transendothelial barrier function (e.g., TBI) is of relevance. In a particular embodiment, provided is an in vitro method for identifying a drug candidate capable of i) increasing in vivo transendothelial barrier integrity (TBI) or ii) decreasing in vivo TBI of endothelial cells (ECs), the method consisting of the sequential the steps of:
a) providing ECs capable of establishing high transendothelial electrical resistance (TEER);
b) contacting the ECs with the drug candidate and measuring in vitro TEER before and after contacting the ECs with the drug candidate, or contacting the ECs with the drug candidate and measuring in vitro TEER of the ECs contacted with the drug candidate and in parallel measuring in vitro TEER of ECs not contacted with the drug candidate;
wherein (i) a higher in vitro TEER of the ECs contacted with the drug candidate compared with the in vitro TEER of the ECs not contacted with the drug candidate is indicative of a drug capable of increasing in vivo TBI of ECs, and (ii) a lower in vitro TEER of the ECs contacted with the drug candidate compared with the in vitro TEER of the ECs not contacted with the drug candidate is indicative of a drug capable of decreasing in vivo TBI of ECs.

In a further embodiment, provided is an in vitro method for selecting a drug candidate for in vivo application to an individual suffering from a disease associated with disruption or loss of transendothelial barrier integrity (TBI), the method consisting of the sequential the steps of:
a) providing ECs capable of establishing high transendothelial electrical resistance (TEER);
b) contacting the ECs with the drug candidate and measuring in vitro TEER before and after contacting the ECs with the drug candidate, or contacting the ECs with the drug candidate and measuring in vitro TEER of the ECs contacted with the drug candidate and in parallel measuring in vitro TEER of ECs not contacted with the drug candidate; wherein a drug candidate with a higher in vitro TEER of the ECs contacted with the drug candidate compared with the in vitro TEER of the ECs not contacted with the drug candidate is selected for in vivo application of the drug candidate.

The ECs capable of establishing high TEER as described herein and as used in methods for identifying a drug candidate comprise one or more of the expression vectors as herein described. In one embodiment, the ECs in step a) of the herein described method for selecting a drug candidate are provided as a monolayer of cells, in particular as a confluent monolayer of cells. A confluent monolayer of ECs comprising at least one expression vector comprising nucleic acid encoding at least one transcription factor selected from the group consisting of TAL1, SOX18, FOXF2, SOX7, FOXC1, ETS1, KLF11, LMO2, and LEF1 is capable of establishing high TEER. In a particular embodiment, the cells forming the confluent monolayer express the transcription factors ETS1, SOX18 and SOX7. In preferred embodiments, the cells express the transcription factors i) ETS1, SOX18, SOX7 and TAL1; or (ii) ETS1, SOX18, SOX7 and LEF1, most preferred ETS1, SOX18, SOX7 and TAL1.

In the context of the present invention, a parameter correlating with TBI, e.g., TEER, is measured for a cell culture of interest (e.g., the EC culture contacted with a drug candidate) in comparison to a cell culture at reference conditions (e.g., the EC culture not contacted with a drug candidate). In one embodiment, the measured in vitro TEER of the EC culture contacted with the drug candidate is higher compared to the measured in vitro TEER of the EC culture not contacted with the drug candidate, in particular at least about 1.1-fold, 1.2-fold, 1.3-fold, 1.4-fold, 1.5-fold, 1.6-fold, 1.7-fold, 1.8-fold, 1.9-fold, 2-fold, 5-fold or 10-fold higher compared to the measured in vitro TEER of the EC culture not contacted with the drug candidate. In one embodiment, the measured in vitro TEER of the EC culture contacted with the drug candidate is lower compared to the measured in vitro TEER of the EC culture not contacted with the drug candidate, in particular at least about 1.1-fold, 1.2-fold, 1.3-fold, 1.4-fold, 1.5-fold, 1.6-fold, 1.7-fold, 1.8-fold, 1.9-fold, 2-fold, 5-fold or 10-fold lower compared to the measured in vitro TEER of the EC culture not contacted with the drug candidate.

TEER can be measured by methods known in the art and as provided herein. In one embodiment, TEER is measured in real-time. In one embodiment, the complex impedance spectrum (Z, R, C) of a confluent monolayer of the cells is measured on gold electrodes. In one embodiment, TEER is measured in real-time in a controlled gas environment. In one embodiment, TEER is measured on a multielectrode array. In one embodiment, TEER is measured on an 8-well, a 16-well, a 24-well or a 96-well multielectrode array, preferably on a 96-well multielectrode array. In one embodiment, TEER is measured on an ECIS Z-theta system from Applied Biophysics, preferably on a 96-well multielectrode array.

Accordingly, one embodiment is the use of the EC cultures obtained by the methods according to the invention to determine the efficacy of a drug candidate, in particular in a high-throughput mode. The cultures can be derived from healthy individuals and/or from diseased individuals and results from efficacy and/or toxicity studies performed using the EC cultures as described herein can be integrated to predict disease and/or therapy relevant physiological effects of a drug candidate. In one embodiment, the in vitro efficacy profile of a drug candidate is assessed and drug candidates with favorable efficacy profile are selected for further development. Further development may comprise in vivo testing of the drug candidate in non-human primate species and/or in vivo testing in humans.

### EXEMPLARY EMBODIMENT

1. A method for producing cells capable of establishing high transendothelial electrical resistance (TEER), comprising the step of contacting the cells with at least one transcription factor, wherein a confluent monolayer of the cells establishes higher transendothelial electric resistance compared to a confluent monolayer of cells not contacted with the at least one transcription factor.
2. The method of embodiment 1, wherein the at least one transcription factor is individually selected from the group consisting of TAL1, SOX18, FOXF2, SOX7, FOXC1, ETS1, KLF11, LMO2, and LEF1.
3. The method of any one of embodiments 1 or 2, wherein the at least one transcription factor is selected from the group consisting of ETS1, SOX18 and SOX7.
4. The method of any one of embodiments 1 to 3, wherein the transcription factors are i) ETS1, SOX18, SOX7 and TAL1; or (ii) ETS1, SOX18, SOX7 and LEF1.
5. The method of any one of embodiments 1 to 4, wherein isolated nucleic acids encoding the at least one transcription factor are introduced into the cell.
6. The method of embodiments 5, wherein the isolated nucleic acids encode the transcription factors ETS1, SOX18 and SOX7.
7. The method of any one of embodiments 5 or 6, where the isolated nucleic acids encode the transcription factors (i) ETS1, SOX18, SOX7 and TAL1; or (ii) ETS1, SOX18, SOX7 and LEF1.
8. The method of any one of embodiments 1 to 7, wherein the isolated nucleic acids are comprised in at least one expression vector.
9. The method of embodiment 8, wherein the at least one expression vector is individually selected from the group consisting of a viral vector, a non-viral vector, and a plasmid vector.
10. The method of any one of embodiments 1 to 9, wherein 1, 2, 3, or 4 expression vectors are introduced into the cell.
11. The method of any one of embodiments 1 to 10, wherein the cell is a mammalian cell, in particular a human cell.
12. The method of any one of embodiment 1 to 11, wherein the cell is an endothelial cell (EC).
13. The method of any one of embodiments 1 to 12, wherein the cell is generated from a pluripotent stem cell, in particular from an embryonic stem cell or an induced pluripotent stem cell.
14. The method of embodiment 13, comprising incubating the pluripotent stem cells in a priming medium supplemented with a small molecule that activates the Beta-catenin and/or Wnt signaling and/or Hedgehog (HH) signaling and inducing differentiation by incubating the primed cells in an induction medium.
15. The method of embodiment 14, wherein the small molecule that activates the Beta-catenin and/or Wnt signaling and/or Hedgehog (HH) signaling is selected from the group consisting of small molecule inhibitors of glycogen synthase kinase 3 (Gsk3a-b), small molecule inhibitors of CDC-like kinase 1 (Clk1-2-4, small molecule inhibitors of mitogen-activated protein kinase 15 (Mapk15), small molecule inhibitors of dual-specificity tyrosine-(Y)-phosphorylation regulated kinase (Dyrk1a-b 4), small molecule inhibitors of cyclin-dependent kinase 16 (Pctk1-3 4), Smoothened (SMO) activators and modulators of the interaction between β-catenin (or γ-catenin) and the coactivator proteins CBP (CREB binding protein) and p300 (E1A binding protein p300).
16. The method of any one of embodiments 14 or 15, wherein the priming medium is supplemented with a small molecule inhibitor of Transforming growth factor beta (TGF β).
17. The method of embodiment 16, wherein the small molecule inhibitor of TGF β is SB431542.
18. The method of any one of embodiments 14 to 17, comprising incubating the cells in the priming medium for 2 to 4 days, in particular for 3 days.
19. The method of any one of embodiments 14 to 18, wherein the priming medium of step a) is a serum free medium supplemented with insulin, transferrin and progesterone.
20. The method of any one of embodiments 14 to 19, wherein the small molecule that activates the Beta-catenin and/or Wnt signaling and/or Hedgehog (HH) signaling of step a) is 3-(3-Amino-phenyl)-4-(1-methyl-1H-indol-3-yl)-pyrrole-2,5-dione (CP21R7).
21. The method of any one of embodiments 14 to 20, wherein the priming medium of step a) additionally comprises recombinant bone morphogenic protein-4 (BMP4).
22. The method of any one of embodiments 14 to 21, wherein the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-H-indol-3-yl)-pyrrole-2,5-dione) and 10-50 ng/ml recombinant bone morphogenic protein-4 (BMP4), in particular wherein the priming medium comprises 1 μM CP21R7 and 25 ng/ml BMP4.
23. The method of any of embodiments 14 to 22, wherein the induction medium is a serum-free medium supplemented with VEGF-A (Vascular endothelial growth factor) or placenta-like growth factor 1 (PLGF-1) and a small molecule adenylate cyclase activator.
24. The method of embodiment 23, wherein the small molecule adenylate activators is selected from the group comprising Forskolin ((3R)-(6aalphaH)Dodecahydro-6beta,10alpha,10balpha-trihydroxy-3beta,4abeta,7,7,10abeta-pentamethyl-1-oxo-3-vinyl-1H-naphtho[2,1-b]pyran-5beta-ylacetate),8-Bromo-cAMP (8-Bromoadenosine-3′,5′-cyclic monophosphate) and Adrenomedullin.
25. The method of any one of embodiments 14 to 24, wherein the induction medium is a serum-free medium supplemented 1-10 μM Forskolin and 5-100 ng/ml VEGF-A, in particular 200 ng/ml VEGF and 2 μM Forskolin.
26. The method of any one of embodiments 14 to 25, comprising incubating the cells in the induction medium for 18 hours to 48 hours.
27. The method of any one of embodiments 1 to 26, additionally comprising incubating the product of step a) in an expansion medium suitable for proliferation of the ECs.
28. The method of embodiment 27, wherein the expansion medium is supplemented with VEGF-A, in particular with 50 ng/ml VEGF-A.
29. The method of any one of embodiments 13 to 28, wherein the pluripotent stem cells are derived from a subject suffering from a disease associated with vascular complications.
30. The method of any one of embodiments 1 to 29, wherein the isolated nucleic acids comprise at least one polynucleotide.
31. The method of any one of embodiments 1 to 30, additionally comprising freezing, storing and/or re-thawing the cells wherein the cells remain viable.
32. An expression vector comprising isolated nucleic acids encoding at least one transcription factor selected from the group consisting of TAL1, SOX18, FOXF2, SOX7, FOXC1, ETS1, KLF11, LMO2, and LEF1.
33. The expression vector of embodiment 32, wherein the isolated nucleic acids encode the transcription factors ETS1, SOX18 and SOX7.
34. The expression vector of any one of embodiments 32 or 33, wherein the isolated nucleic acids encode the transcription factors (i) ETS1, SOX18, SOX7 and TAL1; or (ii) ETS1, SOX18, SOX7 and LEF1.
35. The expression vector of any one of embodiments 32 to 34 which is selected from the group consisting of a viral vector, a non-viral vector, and a plasmid vector.
36. A kit comprising two or more individual expression vectors of any one of embodiments 32 to 35.
37. The kit of embodiment 36, wherein the expression vectors encode the transcription factors ETS1, SOX18 and SOX7.
38. The kit of any one of embodiments 36 or 37, wherein the expression vectors encode the transcription factors (i) ETS1, SOX18, SOX7 and TAL1; or (ii) ETS1, SOX18, SOX7 and LEF1.
39. A cell comprising the expression vector of any one of embodiments 32 to 35 or the expression vectors of the kit of any one of embodiments 36 to 38.
40. The cell according to embodiment 39, wherein the cell is an endothelial cell.
41. The cell according to anyone of embodiments 39 or 40, wherein the cell is a mammalian cell, in particular a human cell.
42. The cell according to any one of embodiments 39 to 41, wherein the cell is generated from a pluripotent stem cell, in particular from an embryonic stem cell or an induced pluripotent stem cell.
43. The method according to any one of claim 1 to 31, wherein the cells capable of establishing high TEER are used for identifying a drug candidate capable of i) increasing in vivo transendothelial barrier integrity (TBI) or ii) decreasing in vivo TBI of endothelial cells.
44. The method according to claim 43 comprising the steps of:
   (a) providing a monolayer of cells capable of establishing high TEER;
   (b) contacting the cells with the drug candidate;
   (c) measuring in vitro TEER before and after contacting the cells with the drug candidate, or measuring in vitro TEER of the cells contacted with the drug candidate and in parallel measuring in vitro TEER of cells not contacted with the drug candidate;
   wherein (i) a higher in vitro TEER of the cells contacted with the drug candidate compared with the in vitro TEER of the cells not contacted with the drug candidate is indicative of a drug capable of increasing in vivo TBI of ECs, and (ii) a lower in vitro TEER of the cells contacted with the drug candidate compared with the in vitro TBI of the cells not contacted with the drug candidate is indicative of a drug capable of decreasing in vivo TBI of ECs.
45. The method of embodiment 44, wherein the cells capable of establishing high TEER comprise at least one of the expression vector of any one of embodiments 32 to 35 or the expression vectors of any of the kit according to embodiments 36 to 38.
46. The method of any one of embodiments 43 to 45, wherein the cells in step a) are provided on a cell culture support, in particular on a multi-well plate, more particular on a multi-well plate selected from the group consisting of a 24-well plate, a 96-well plate, a 384-well plate, or a 1536-well plate.
47. The method of any one of embodiments 43 to 46, wherein the TEER is indicative for in vitro TBI.
48. The method of any one of embodiments 43 to 47, which is performed in a high-throughput mode.
49. The method of any one of embodiments 43 to 48, which is used to screen drug molecules in a drug development setting, in particular for high-throughput screening of a drug candidate compound library.
50. A method for identifying a drug candidate capable of i) increasing in vivo transendothelial barrier integrity (TBI) or ii) decreasing in vivo TBI of endothelial cells, the method comprising the steps of:
   (a) providing a monolayer of the cells of any one of claims 39 to 42;
   (b) contacting the cells with the drug candidate;
   (c) measuring in vitro TEER before and after contacting the cells with the drug candidate, or measuring in vitro TEER of the cells contacted with the drug candidate and in parallel measuring in vitro TEER of cells not contacted with the drug candidate;
   wherein (i) a higher in vitro TEER of the cells contacted with the drug candidate compared with the in vitro TEER of the cells not contacted with the drug candidate is indicative of a drug capable of increasing in vivo TBI of ECs, and (ii) a lower in vitro TEER of the cells contacted with the drug candidate compared with the in vitro TEER of the cells not contacted with the drug candidate is indicative of a drug capable of decreasing in vivo TBI of ECs.
51. The invention as hereinbefore described.

### Materials and Methods

Human PSC culture and differentiation. The hPSC line SA001 (Englund M C, Caisander G, Noaksson K, Emanuelsson K, Lundin K, Bergh C, et al. In vitro cellular & developmental biology Animal. 2010; 46(3-4):217-30, Cellartis A B) was differentiated as described (Patsch C, Challet-Meylan L, Thoma E C, Urich E, Heckel T, O'Sullivan J F, et al. Nat Cell Biol. 2015; 17(8):994-1003) with some modifications: expansion medium consisting of StemPro with 50 ng/mL of VEGFA has been kept on cells only for the first division. From the second division cells were cultured using VascuLife VEGF Endothelial Medium Complete Kit (LifeLine Cell Technology). Final composition of the supplements added to the media was 10% FBS, 4 mM L-Glutamine, 0.75 U/mL Heparin sulfate, 5 ng/mL FGF-2, 5 ng/mL EGF, 5 ng/mL VEGFA, 15 ng/mL IGF1, 1 μg/mL Hydrocortizone Hemisuccinate, 50 μg/mL Ascorbic acid. SB431542 (10 μM) was supplemented to the media.

Adenovirus Production.

Several promoters (UbiC, E1Fα and CMV) expressing GFP were tested with different multiplicity of infection (MOI) of adenovirus (data not shown). The UbiC promoter was selected for overexpression of the transcription factors at 80 MOI. Downstream of the UbiC promoter followed by the 3′ sequence of GFP with polyadenylation ORF sequence was cloned. Bacterial artificial chromosome was generated by transformation and recombination in Escherichia coli DH10B carrying SIR-BAC-Ad5 encoding an E1- and E3-deficient adenovirus genome. Infectious recombinant adenovirus particles were generated by transfecting linearized construct into HEK293 cells followed by purification of replication-competent adenoviruses using AdenoONE Purification Kit.

Analysis of published gene expression datasets. Normalized count expression from single cell expression data from EC, smooth muscle cells and PDGFRα has been downloaded from GEO (GSE98816, (Vanlandewijck M, He L, Mae M A, Andrae J, Ando K, Del Gaudio F, et al. Nature. 2018; 554(7693):475-80)). Single cell expression violin plots with mean expression of selected transcription factors have been plotted using R package ggplot2. Correlation of gene expression to CLDN5 in single cell expression data was done using Pearson correlation in R. Raw Affymetrix expression files were downloaded from GEO (GSE47067 (Nolan D J, Ginsberg M, Israely E, Palikuqi B, Poulos M G, James D, et al. Developmental cell. 2013; 26(2):204-19), GSE35802 (Tam S J, Richmond D L, Kaminker J S, Modrusan Z, Martin-McNulty B, Cao T C, et al. Developmental cell. 2012; 22(2):403-17), GSE48209 (Coppiello G, Collantes M, Sirerol-Piquer M S, Vandenwijngaert S, Schoors S, Swinnen M, et al. Circulation. 2015; 131(9):815-26)), and analyzed using Partek Genomic Suite using. Probeset data were normalized as follows: pre-background adjustments were made for GC correction and probe sequence bias followed by RMA background correction and quantile normalization. Normalized probeset data were log 2 transformed and summarized by median polish method. Differential expression was determined by an ANOVA model. For the Nolan et al. study (Nolan D J, Ginsberg M, Israely E, Palikuqi B, Poulos M G, James D, et al. Developmental cell. 2013; 26(2):204-19) where the brain endothelial cells were compared to several other tissues, RankProdIt (Laing E, Smith C P. BMC research notes. 2010; 3:221), a rank based meta-analysis tool, to generate a consensus brain ECs gene signature was used.

Cell lysis and RNA isolation. Cultured ECs were lysed using 350 μL RLT lysis buffer (Qiagen)+β-mercaptoethanol (1%) and subsequently vortexed for 1 min at room temperature and snap frozen. RNA was isolated from cell lysates using automated Maxwell Total RNA purification kit (Promega) with DNA removal with DNAse I digestion.

RNA-sequencing and analysis. Total RNA was subjected to oligo (dT) capture and enrichment, and the resulting mRNA fraction was used to construct ccDNA libraries. Sequencing was performed on the Illumina HiSeq platform using the standard protocol (TruSeq Stranded Total RNA Library, Illumina) that generated approximately 30 million reads of 50 base-pair per sample. Reads were mapped to the human genome (hg19/Refseq) using STAR (Dobin A, Davis C A, Schlesinger F, Drenkow J, Zaleski C, Jha S, et al. Bioinformatics. 2013; 29(1):15-21) and counting was performed using union mode of HtSeq (Anders S, Pyl P T, Huber W. Bioinformatics. 2015; 31(2):166-9). Differential expression was performed using DESeq2 (Dobin A, Davis C A, Schlesinger F, Drenkow J, Zaleski C, Jha S, et al. Bioinformatics. 2013; 29(1):15-21). Gene set enrichment analysis was performed using GSEA (Subramanian A, Tamayo P, Mootha V K, Mukherjee S, Ebert B L, Gillette M A, et al. Proceedings of the National Academy of Sciences of the United States of America. 2005; 102(43):15545-50) using Hallmarks MsigDb database (Liberzon A, Birger C, Thorvaldsdottir H, Ghandi M, Mesirov J P, Tamayo P. Cell Syst. 2015; 1(6):417-25) using weighted analysis with gene list sorted on fold change from upregulated to downregulated genes, following default conditions with ignoring gene sets smaller than 15 and larger than 500 genes.

### Example 1

To identify transcription factors that can differentiate hPSC-ECs to ECs of high transendothelial barrier resistance published expression datasets of the different mouse vascular beds were analyzed (Tam S J, Richmond D L, Kaminker J S, Modrusan Z, Martin-McNulty B, Cao T C, et al. Developmental cell. 2012; 22(2):403-17., Coppiello G, Collantes M, Sirerol-Piquer M S, Vandenwijngaert S, Schoors S, Swinnen M, et al. Circulation. 2015; 131(9):815-26, Nolan D J, Ginsberg M, Israely E, Palikuqi B, Poulos M G, James D, et al. Developmental cell. 2013; 26(2):204-19). Fold change expression in studies between brain ECs and ECs of other vascular beds for all transcription factors (Ravasi T, Suzuki H, Cannistraci C V, Katayama S, Bajic V B, Tan K, et al. Cell. 2010; 140(5):744-52) was calculated. Transcription factors that were highly upregulated (>2 FC) in ECs in brain were identified. Next, gene expression data from a recent single cell expression profiling study from brain ECs (1445 cells analyzed (Vanlandewijck M, He L, Mae M A, Andrae J, Ando K, Del Gaudio F, et al. Nature. 2018; 554(7693):475-80)) was reanalyzed and high expression of CLDN5, an important tight junction that mediates endothelial cell barrier resistance was identified. The gene expression of CLDN5 was correlated with other genes in single cell brain ECs in order to identify gene signatures that can be used to predict active transcription factors. The genes with highest correlation were used to predict the activity of transcription factors using Transfac and Jasper analysis which is integrated in enrichR. hPSC-ECs were transduced with single selected transcription factors (adenovirus-80 MOI) and transendothelial resistance was measured using ECIS. Significant induction of EC barrier was observed with overexpression of TAL1 and SOX18 and tendency of increase in barrier for FOXF2, SOX7, FOXC1, ETS1, KLF11, LMO2 and LEF1 at 24 h post barrier stabilization (FIG. 1A). TAL1 has the fastest action on barrier resistance followed by SOX18 (FIG. 1B). ETS1 was able to induce barrier but with slower kinetics (FIG. 1B). Using FITC-dextran permeability assay the strongest barrier activity was shown for SOX18, SOX7, ETS1 and LEF1 (FIG. 1C). No effect was observed for TAL1, as the assay was performed 48 h post-treatment when TAL1 effects have already decreased (FIG. 1C). RNA-seq analysis was performed at 48 h post-treatment and high upregulation of overexpressed transcription factors was measured. Next, GSEA analysis using Hallmarks MsgDB database was performed with focus on pathways relevant for EC barrier induction. Enrichment of Hedgehog signaling was observed for all transcription factors and Wnt signaling upon overexpression of all transcription factors but KLF11. ETS1 and FOXF2 have induced the largest number of pathways suggesting that act up-stream of other transcription factors. In addition, ETS1 and TALT were able to promote broadly angiogenesis. KLF11 was only transcription factor that was able to induce proliferation. Next we focused on individual members of pathways. SOX18 and SOX7 were often inducing same genes (Wnt, Hedgehog and Notch signaling) suggesting that they act in the same network. Focus on marker ECs genes revealed that KLF11, FOXC1 and KLF11 downregulated VEGFR2, VEGFR and CD34 (FIGS. 1D, 1E, 1F) suggesting that they might not be suitable for EC barrier induction as they change the EC phenotype. Next, expression of tight junctions involved in formation of transendothelial cell barrier was analyzed. Downregulation of VE-Cadherin by FOXF2, FOXC1 and KLF11 was observed, while SOX18, TAL1, SOX7 and LEF1 were able to induce it. SOX7 were able to induce PECAM, GJA1, ESAM and JAM2 (FIGS. 1H, 1I, 1H, 1M). SOX18 was able to induce expression of JAM2 and MARVELD2 (FIGS. 1M, 1O). ETS1 was the only transcription factors that was able to induce CLDN5 expression (FIG. 1N). High-resistance barrier shows low expression of PLVAP (Zhou Y, Nathans J. 2014; 31(2):248-56) and high expression of MFSD2A (Ben-Zvi A, Lacoste B, Kur E, Andreone B J, Mayshar Y, Yan H, et al. Nature. 2014; 509:507) and TNFRSF21 (Tam S J, Richmond D L, Kaminker J S, Modrusan Z, Martin-McNulty B, Cao T C, et al. Death receptors DR6 and TROY regulate brain vascular development. Developmental cell. 2012; 22(2):403-17). The data presented herein shows that SOX18, SOX7 and TAL strongly downregulate PLVAP (FIG. 1P). MFSD2A was strongly induced by SOX18 while TNFRSF21 was induced by SOX18, TAL, FOXC1, ETS1 and LEF1 (FIGS. 1Q, 1R).

### Example 2

As transcription factors work in networks (Neph S, Stergachis A B, Reynolds A, Sandstrom R, Borenstein E, Stamatoyannopoulos J A. Cell. 2012; 150(6):1274-86) we have explored possibility of using lower levels of transcription factors (20 MOI) in a combination with other factors. First we had overexpressed single transcription factors at 20 MOI and evaluated resistance. We could not observe any significant induction after 24 h post stabilization of the resistance (FIG. 2A). In the real time ECIS measurements no significant induction of the barrier resistance was observed, only tendency of SOX18 for barrier induction. In FITC-dextran assay SOX18 caused significant reduction of EC barrier permeability, while TAL1 was found to increase permeability as the effect has been reduced by 48 h (FIG. 2B). Next transcription factors were combined that showed to induce barrier either as single factors (SOX18, TAL1) or were able to induce genes involved in formation of EC barrier (ETS1, LEF1, SOX7). The resistance was measured at 24 h post stabilization of resistance by combing fixed factors with LEF1 or TAL1 and a significant increase of barrier resistance was observed for both combinations (FIG. 2C). ECIS in real time showed that combination of ETS1+SOX18+SOX7+TAL1 was able to induce synergistically barrier. The highest induction was 1.7 fold which is higher than additive combination of 4 factors (20 MOI) and also higher than overexpression of single factors with 80 MOI. A significant reduction of permeability in FITC-dextran was observed with both combinations. Finally, qRT-PCR was performed which identified that combinations of transcription factors is able to potently induce Wnt signaling (upregulation of MARVEKD2, TSPAN12, TNFRSF19 and AXIN2 and downregulation of APCDD1; data not shown).

Combinatorial interaction among transcription factors is critical in differentiation of cell types (Ravasi T, Suzuki H, Cannistraci C V, Katayama S, Bajic V B, Tan K, et al. Cell. 2010; 140(5):744-52, Neph S, Stergachis A B, Reynolds A, Sandstrom R, Borenstein E, Stamatoyannopoulos J A. Cell. 2012; 150(6):1274-86). In this work a combination of transcription factors was identified that act synergistically to induce EC barrier resistance. Current models that exist for modeling EC barrier in vitro are highly sophisticated and difficult to accurately reproduce, making them difficult to adapt for drug discovery. Simple combination of 4 transcription factors can be used to generate an in vitro model of ECs of high-barrier resistance that can be used to find novel pathways and molecular targets for treatment of diseases with ECs disruption.

1. A method for producing cells capable of establishing high transendothelial electrical resistance (TEER), comprising the step of contacting the cells with at least one transcription factor, wherein a confluent monolayer of the cells establishes higher transendothelial electric resistance compared to a confluent monolayer of cells not contacted with the at least one transcription factor.

2. The method of claim 1, wherein the at least one transcription factor is individually selected from the group consisting of TAL1, SOX18, FOXF2, SOX7, FOXC1, ETS1, KLF11, LMO2, and LEF1.

3. The method of any one of claim 1 or 2, wherein the at least one transcription factor is selected from the group consisting of ETS1, SOX18 and SOX7.

4. The method of any one of claims 1 to 3, wherein the transcription factors are i) ETS1, SOX18, SOX7 and TAL1; or (ii) ETS1, SOX18, SOX7 and LEF1.

5. The method of any one of claims 1 to 4, wherein isolated nucleic acids encoding the at least one transcription factor are introduced into the cell.

6. The method of claim 5, wherein the isolated nucleic acids are comprised in at least one expression vector, in particular wherein the at least one expression vector is individually selected from the group consisting of a viral vector, a non-viral vector, and a plasmid vector.

7. The method of any one of claim 1 to 6, wherein the cell is an endothelial cell (EC).

8. An expression vector comprising isolated nucleic acids encoding at least one transcription factor selected from the group consisting of TAL1, SOX18, FOXF2, SOX7, FOXC1, ETS1, KLF11, LMO2, and LEF1

9. The expression vector of claim 8, which is a viral vector, a non-viral vector, or a plasmid vector.

10. The expression vector of any one of claim 8 or 9, wherein the isolated nucleic acids encode the transcription factors ETS1, SOX18 and SOX7.

11. The expression vector of any one of claims 8 to 10, wherein the isolated nucleic acids encode the transcription factors (i) ETS1, SOX18, SOX7 and TAL1; or (ii) ETS1, SOX18, SOX7 and LEF1.

12. A cell comprising one or more of the expression vectors of any one of claims 8 to 11.

13. The cell according to claim 12, wherein the cell is a mammalian cell, in particular a human cell.

14. The method according to any one of claim 1 to 7, wherein the cell capable of establishing high TEER is used for identifying a drug candidate capable of i) increasing in vivo transendothelial barrier integrity (TBI) or ii) decreasing in vivo TBI of endothelial cells.

15. The method according to claim 14 comprising the steps of:
(a) providing a monolayer of cells capable of establishing high TEER;
(b) contacting the cells with the drug candidate;
(c) measuring in vitro TEER before and after contacting the cells with the drug candidate, or measuring in vitro TEER of the cells contacted with the drug candidate and in parallel measuring in vitro TEER of cells not contacted with the drug candidate;
wherein (i) a higher in vitro TEER of the cells contacted with the drug candidate compared with the in vitro TEER of the cells not contacted with the drug candidate is indicative of a drug capable of increasing in vivo TBI of ECs, and (ii) a lower in vitro TEER of the cells contacted with the drug candidate compared with the in vitro TEER of the cells not contacted with the drug candidate is indicative of a drug capable of decreasing in vivo TBI of ECs.

16. A method for identifying a drug candidate capable of i) increasing in vivo transendothelial barrier integrity (TBI) or ii) decreasing in vivo TBI of endothelial cells, the method comprising the steps of:
(a) providing a monolayer of the cells of any one of claim 12 or 13;
(b) contacting the cells with the drug candidate;
(c) measuring in vitro TEER before and after contacting the cells with the drug candidate, or measuring in vitro TEER of the cells contacted with the drug candidate and in parallel measuring in vitro TEER of cells not contacted with the drug candidate;
wherein (i) a higher in vitro TEER of the cells contacted with the drug candidate compared with the in vitro TEER of the cells not contacted with the drug candidate is indicative of a drug capable of increasing in vivo TBI of ECs, and (ii) a lower in vitro TEER of the cells contacted with the drug candidate compared with the in vitro TEER of the cells not contacted with the drug candidate is indicative of a drug capable of decreasing in vivo TBI of ECs.
